(19) United States
(12) Patent Application Publication
(10) Pub. No.: US 2003/0152947 A1
(43) Pub. Date: Aug. 14, 2003

(54) Methods for detecting and treating the early onset of aging-related conditions

(76) Inventors: David C. Crossman, South Yorkshire (GB); Gordon W. Duff, South Yorkshire (GB); Sheila E. Francis, South Yorkshire (GB); Katherine Stephenson, Helotes, TX (US); Kenneth S. Kornman, Newton, MA (US)

Correspondence Address: FOLEY HOAG, LLP, PATENT GROUP, WORLD TRADE CENTER WEST, 155 SEAPORT BLVD, BOSTON, MA, 02110, US

(21) Appl. No.: 10/172,919

(22) Filed: Jun. 17, 2002

Related U.S. Application Data
(60) Provisional application No. 60/298,493, filed on Jun. 15, 2001.

(51) Int. Cl. C12Q 1/68

(52) U.S. Cl. 435/6

(57) ABSTRACT

Certain aspects of the invention relate to methods for determining a subject's susceptibility to the early onset or progression of aging-related conditions. In certain aspects the invention relates to accessing the genotype of a subject with respect to an allele of IL-1 pattern 1, pattern 2 and/or pattern 3. In other aspects, the invention relates to methods for selecting a therapeutic regimen, identifying age-related biomarkers, monitoring the progress of age-related conditions and identifying therapeutics for delaying or diminishing the onset of aging-related conditions.

### CROSS-REFERENCE TO RELATED APPLICATIONS

This application claims priority to U.S. Provisional Application No. 60/298,493, filed Jun. 15, 2001 and incorporated by reference herein in its entirety.

### BACKGROUND

Cellular and Genetic Causes of Aging

Aging, also known as senescence, is a time-dependent degradation of the function of various organs and tissues in the body. While all people eventually experience the effects of aging, the rate at which aging occurs varies tremendously from individual to individual. The variation in the rate of aging is caused by a mixture of environmental and genetic causes. Studies of survivorship and longevity in humans, as well as animals, demonstrate a strong heritable component to the aging process. In addition, several well-known genetic disorders cause individuals to undergo what appears to be premature aging. For example, individuals with Werner's syndrome experience various symptoms of aging in their teenage years and usually die before age 30 (Yu et al. 1996 Science 272:258; Ye et al. 1997 Am. J. Med. Genet. 68:4940).

Aging has a cellular correlate which is likely to form part of the mechanism of aging in the whole organism. Aging-related phenomena can be viewed as a result of diminished or abnormal function in particular cell types. For example, senescence may result from any of the following changes at the cellular level: increased likelihood of apoptosis, inability to undergo further cell division, mutations in the cellular repair systems, and altered gene expression (which may result from a variety of causes, such as for example, growth hormone or sex hormone withdrawal or the accumulation of reactive oxygen end-products and glycated endproducts). Thus, although aging is a process that affects the whole organism, there is growing evidence that events occurring within a single cell or cell culture can be an important causative factor or marker for the aging process.

Wrinkled skin in an example of an aging process that has been related to cellular events. Wrinkled skin is a common condition in aging individuals. Wrinkled skin results primarily from the decreasing ability of cells such as fibroblasts to repair damage to the skin, and particularly to repair damage to the collagen fibers that provide much of the structural support for skin. A recent whole genome analysis of aging fibroblasts demonstrated that these cells have perturbed expression of a number of genes required for generating and remodeling collagen fibers and the extracellular matrix (Ly et al. (2000) Science 287:2486). Similar cellular mechanisms may underlie the effects of aging on aspects of human biology ranging from the immune system to neural function.

Normal human cells have a finite life-span determined by the number of replications the cell can undergo. When cells enter a non-dividing state they exhibit changes that appear to influence age-related pathologies of the whole organism. For example, homeostatic remodeling of tissues, such as structural elements of the body including skin, bone, and connective tissues, requires replication of specific cells, such as fibroblasts and osteoblasts. In any segment of tissue at a specific time, a certain proportion of the cells are replicating. That proportion increases or decreases based on the stimuli and biochemical regulatory process within the tissue. If fewer cells are capable of responding to the replication signals, the tissue segment will be compromised in its ability to regenerate and remodel. For example, bone is constantly remodeling without an overt loss of structural integrity during the first 4-5 decades. This occurs due to a tight linkage between osteoclastic activity that destroys bone and osteoblastic activity that builds bone. A discoordination of the two processes, such as a minor reduction in the ability of the osteoblastic activity to respond would result in a loss of bone integrity.

A process similar, in some ways, to remodeling occurs with normal wound healing. When some damage or insult occurs in a tissue, the body responds with a programmed series of cell migrations, replications, and specific gene expression at the site of injury, resulting in repair and regeneration of the damaged tissue. A major component of this process is cell replication as the site of injury. If there is a reduction in the population of cells capable of replicating, there will be some compromise in the final integrity of the healed area.

Even when cells are not replicating, they are involved in tissue function by expressing chemicals that participate in the regulation and response of that tissue. This is the “phenotypic expression” of the cell and is a result of selective and orchestrated gene expression. It has been established that senescent cells have altered patterns of gene expression and therefore are likely to alter tissue function and, ultimately, the functioning of the body. Such an alteration of normal cell function may explain the inability of aging cells to remove toxic wastes, resulting in accumulations of calcified products and amyloid which often characterize diseases of aging.

Non-transformed cells, when grown in culture with the appropriate growth factors and extracellular matrix, will divide and continue to do so for some time, but growth in culture, eventually ceases (Hayflick L. (1975) Fed. Proc. 34:9-13). This phenomenon now appears to be an innate part of cell biology and arises from repeated divisions of the cell (mitotic time) rather than the accumulation of environmental damage or the depletion of essential trace elements or nutrients (chronological or metabolic time) (Dell'Orco et al. (1973) 77:356-60; Harley et al. (1978) J. Cell Physiol. 97:509-16). The mitotic clock has been hypothesized to be telomere length (Harley CB (1991) Mutat. Res. 256:271-82). This mechanism, by which cells count their divisions, appears to be not only a counting mechanism but also a part of the signal for a cellular senescence program. In non-transformed, somatic cells, which lack the enzyme telomerase, continuous division of cells leads to a progressive shortening of telomere length. Such shortening of the chromosomes eventually impairs further DNA synthesis, and the end of life in culture is characterized by G1/S phase arrest. Transfection of the catalytic subunit of telomerase into non-transformed cells has been shown to extend life in culture (Bodnar et al. (1998) Science 279:349-52). Telomeric shortening occurs during the aging process; however significant interindividual variation exists relative to age-induced shortening of telomeres suggesting environmental and genetic factors most likely play a role in this process.

Telomeric shortening and subsequent replicative senescence may reflect cumulative oxidative damage to cells. Age-dependent telomere shortening is attenuated in vitro by the addition of an oxidation-resistant form of ascorbic acid. This process may play a significant role for the aging of the immune system. In addition, increased telomeric instability and reduced growth potential has been associated with ovarian carcinogenesis in ovarian surface epithelium from females with a family history of breast or ovarian cancer. The accumulation of genetic aberrations due to accelerated cellular senescence may contribute to enhanced susceptibility for malignant transformation and metastasis. Finally, telomeric shortening may have an important role in senescence of vascular endothelial cells and atherosclerotic disease progression. In cultured endothelial cells, telomerase activity decreases prior to the development of a senescent phenotype, and intriguingly, nitric oxide, an important atheroprotective factor, significantly reduces endothelial cell senescence and age-dependent inhibition of telomerase activity (Vasa et al. (2000) Circulation Res. 87: 540-542).

In cell culture the mitotic age of cells is measured by cumulative population doublings (CPDs). The number of CPDs at end of life in culture is, to some extent, cell type specific. This variation could reflect the number of divisions that have occurred prior to the cell culture, or it there may indicate the action of other cell specific mechanisms. The relationship between the cellular senescence program and aging of the whole organism remains poorly understood. Fibroblasts cultured from older individuals undergo fewer CPDs but the relationship is not precise. Additionally, the mean telomere length of human vascular endotheral cells decrease with the increasing age of the donor, and cells cultured from arterial plaques evince decreased representative lifespans (Chang et al. (1995) PNAS 92:11190-11194). In general, it appears that cells that are approaching end of life in culture display phenotypic changes that are plausibly linked to the aging process as a whole.

Genetics of the IL-1 Gene Cluster

The IL-1 gene cluster is on the long arm of chromosome 2 (2q13) and contains at least the genes for IL-1α (IL-1A), IL-1β (IL-1B), and the IL-1 receptor antagonist (IL-1RN), within a region of 430 Kb (Nicklin, et al. (1994) Genomics, 19: 382-4). The agonist molecules, IL-1α and IL-1β, have potent pro-inflammatory activity initiate many inflammatory cascades. Their actions, often via the induction of other cytokines such as IL-6 and IL-8, lead to activation and recruitment of leukocytes into damaged tissue, local production of vasoactive agents, fever response in the brain and hepatic acute phase response. All three IL-1 molecules bind to type I and to type II IL-1 receptors with varying affinities, but only the type I receptor transduces a signal to the interior of the cell. In contrast, the type II receptor is shed from the cell membrane and acts as a decoy receptor. The receptor antagonist and the type II receptor, therefore, are both anti-inflammatory in their actions.

Certain alleles from the IL-1 gene cluster are already known to be associated with particular disease states. For example, IL-1RN allele 2 has been shown to be associated with coronary artery disease (PCT/US/98/04725, and U.S. Ser. No. 08/813,456), osteoporosis (U.S. Pat. No. 5,698,399), nephropathy in diabetes mellitus (Blakemore, et al. (1996) Hum. Genet. 97(3): 369-74), alopecia areata (Cork, et al., (1995) J. Invest. Dermatol. 104(5 Supp.): 15S-16S; Cork et al. (1996) Dermatol Clin 14: 671-8), Graves disease (Blakemore, et al. (1995) J. Clin. Endocrinol. 80(1): 111-5), systemic lupus erythematosus (Blakemore, et al. (1994) Arthritis Rheum. 37: 1380-85), lichen sclerosis (Clay, et al. (1994) Hum. Genet 94: 407-10), and ulcerative colitis (Mansfield, et al. (1994) Gastoenterol. 106(3): 637-42).

In addition, the IL-1A allele 2 from marker −889 and IL-1B (TaqI) allele 2 from marker+3954 have been found to be associated with periodontal disease (U.S. Pat. No. 5,686,246; Komman and diGiovine (1998) Ann Periodont 3: 327-38; Hart and Kornman (1997) Periodontol 2000 14: 202-15; Newman (1997) Compend Contin Educ Dent 18: 881-4; Kornnan et al. (1997) J. Clin Periodontol 24: 72-77). The IL-1A allele 2 from marker −889 has also been found to be associated with juvenile chronic arthritis, particularly chronic iridocyclitis (McDowell, et al. (1995) Arthritis Rheum. 38: 221-28). The IL-1B (TaqI) allele 2 from marker +3954 of IL-1B has also been found to be associated with psoriasis and insulin dependent diabetes in DR3/4 patients (di Giovine, et al. (1995) Cytokine 7: 606; Pociot, et al. (1992) Eur J. Clin. Invest. 22: 396-402). Additionally, the IL-1RN (VNTR) allele 1 has been found to be associated with diabetic retinopathy (see U.S. Ser. No. 09/037,472, and PCT/GB97/02790). Furthermore allele 2 of IL-1RN (VNTR) has been found to be associated with ulcerative colitis in Caucasian populations from North America and Europe (Mansfield, J. et al., (1994) Gastroenterology 106: 637-42). Interestingly, this association is particularly strong within populations of ethnically related Ashkenazi Jews (PCT WO97/25445).

Genotype Screening

Traditional methods for the screening of heritable diseases have depended on either the identification of abnormal gene products (e.g., sickle cell anemia) or an abnormal phenotype (e.g., mental retardation). These methods are of limited utility for heritable diseases with late onset and no easily identifiable phenotypes such as, for example, a predisposition to early aging. With the development of simple and inexpensive genetic screening methodology, it is now possible to identify polymorphisms that indicate a propensity to develop disease, even when the disease is of polygenic origin. The number of diseases that can be screened by molecular biological methods continues to grow with increased understanding of the genetic basis of multifactorial disorders.

Genetic screening (also called genotyping or molecular screening), can be broadly defined as testing to determine if a patient has mutations (or alleles or polymorphisms) that either cause or alter a disease state or are “linked” to the mutation causing or altering a disease state. Linkage refers to the phenomenon that DNA sequences which are close together in the genome have a tendency to be inherited together. Two sequences may be linked because of some selective advantage of co-inheritance. More typically, however, two polymorphic sequences are co-inherited because of the relative infrequency with which meiotic recombination events occur within the region between the two polymorphisms. The co-inherited polymorphic alleles are said to be in linkage disequilibrium with one another because, in a given human population, they tend to either both occur together or else not occur at all in any particular member of the population. Indeed, where multiple polymorphisms in a given chromosomal region are found to be in linkage disequilibrium with one another, they define a quasi-stable genetic “haplotype.” In contrast, recombination events occurring between two polymorphic loci cause them to become separated onto distinct homologous chromosomes. If meiotic recombination between two physically linked polymorphisms occurs frequently enough, the two polymorphisms will appear to segregate independently and are said to be in linkage equilibrium.

While the frequency of meiotic recombination between two markers is generally proportional to the physical distance between them on the chromosome, the occurrence of “hot spots” as well as regions of repressed chromosomal recombination can result in discrepancies between the physical and recombinational distance between two markers. Thus, in certain chromosomal regions, multiple polymorphic loci spanning a broad chromosomal domain may be in linkage disequilibrium with one another, and thereby define a broad-spanning genetic haplotype. Furthermore, where a disease-causing mutation is found within or in linkage with this haplotype, one or more polymorphic alleles of the haplotype can be used as a diagnostic or prognostic indicator of the likelihood of developing the disease. This association between otherwise benign polymorphisms and a disease-causing polymorphism occurs if the disease mutation arose in the recent past, so that sufficient time has not elapsed for equilibrium to be achieved through recombination events. Therefore identification of a human haplotype which spans or is linked to a disease-causing mutational change, serves as a predictive measure of an individual's likelihood of having inherited that disease-causing mutation. Importantly, such prognostic or diagnostic procedures can be utilized without necessitating the identification and isolation of the actual disease-causing lesion. This is significant because the precise determination of the molecular defect involved in a disease process can be difficult and laborious, especially in the case of multifactorial diseases such as inflammatory disorders.

Indeed, the statistical correlation between a disorder and an IL-1 polymorphism does not necessarily indicate that the polymorphism directly causes the disorder. Rather the correlated polymorphism may be a benign allelic variant which is linked to (i.e. in linkage disequilibrium with) a disorder-causing mutation which has occurred in the recent human evolutionary past, so that sufficient time has not elapsed for equilibrium to be achieved through recombination events in the intervening chromosomal segment. Thus, for the purposes of diagnostic and prognostic assays for a particular disease, detection of a polymorphic allele associated with that disease can be utilized without consideration of whether the polymorphism is directly involved in the etiology of the disease. Furthermore, where a given benign polymorphic locus is in linkage disequilibrium with an apparent disease-causing polymorphic locus, still other polymorphic loci which are in linkage disequilibrium with the benign polymorphic locus are also likely to be in linkage disequilibrium with the disease-causing polymorphic locus. Thus these other polymorphic loci will also be prognostic or diagnostic of the likelihood of having inherited the disease-causing polymorphic locus. Indeed, a broad-spanning human haplotype (describing the typical pattern of co-inheritance of alleles of a set of linked polymorphic markers) can be targeted for diagnostic purposes once an association has been drawn between a particular disease or condition and a corresponding human haplotype. Thus, the determination of an individual's likelihood for developing a particular disease of condition can be made by characterizing one or more disease-associated polymorphic alleles (or even one or more disease-associated haplotypes) without necessarily determining or characterizing the causative genetic variation.

Given that the onset of aging-related conditions is, in part, determined by genetic factors, it would be desirable to identify these genetic factors and develop methods of identifying such factors in an individual.

### SUMMARY

In general, the invention relates to the observation that certain IL-1 genotypes are indicators for a genetic influence on aging and the mechanistically related process of cellular senescence. In certain aspects, the present application relates to methods for determining a subject's susceptibility to the early onset or progression of an aging-related conditions (EOA). In one aspect, a method of the invention comprises obtaining a nucleic acid sample from a subject, and testing for the presence of at least one EOA-associated allele of an IL-1 haplotype, such as pattern 1, pattern 2 and/or pattern 3.

In certain embodiments, aging-related conditions of the invention include impaired connective tissue function, cardiovascular disease, age-related cancer, abnormal immune system function and impaired neurological function. In additional embodiments, aging-related conditions of the invention are conditions that result, at least in part, from the accumulation of calcified products and amyloid, oxidative damage or increased production of reactive oxygen species. In preferred embodiments, aging-related conditions are in part the result of cellular senescence, including increased apoptosis, decreased ability to undergo cell division, mutations in cellular repair systems, and changes in cell behavior caused by the buildup of undesired by-products, such as byproducts of oxidative damage or glycation. Furthermore, many of the aforementioned conditions are fatal, and it is therefore understood that methods of the invention may be used to detect the of a shorter average life span.

In preferred embodiments, age-related conditions of the invention include the following: osteoporosis, osteoarthritis, decreased chondrocyte proteoglycan synthesis, decreased wound healing, wrinkled skin, rheumatoid arthritis, amyloidosis, Alzheimer's disease, type 2 diabetes mellitus, reduced T cell proliferation, increased IL-1 production, decreased responsiveness to IL-1, decreased resistance to infection, impaired long-term potentiation in hippocampal neurons, decreased synaptic plasticity, memory loss, hearing loss, changes in the eye, including but not limited to retinal degeneration. depression, insomnia, impaired learning, endometrial cancer, prostate cancer, ovarian cancer, breast cancer, coronary artery disease, cerebrovascular disease (such as, but not limited to, stroke), peripheral artery disease, atherosclerosis, congestive heart failure and hypertension.

In a further aspect, the invention relates to conditions related to the population doubling of endothelial cells. In certain embodiments, the presence of an IL-1 allele of pattern 1 or pattern 2 is indicative of a decreased potential for cell division in endothelial cells. Such decreased potential may be associated with a wide array of conditions and disorders. For example, the process of angiogenesis requires cell division of endothelial cells to form a tube that becomes a new blood vessel. Angiogenesis occurs in a range of disease states, tumor metastases and abnormal growths by endothelial cells. The vasculature created as a result of angiogenic processes supports the pathological damage seen in these conditions. The diverse pathological states created due to unregulated angiogenesis have been grouped together as angiogenic dependent or angiogenic associated diseases. Angiogenesis is also involved in normal processes such as wound healing and reproduction.

In a further embodiment, preferred alleles of the pattern 1 haplotype include: the IL-1A (222/223) allele 3, IL-1A (gz5/gz6) allele 3, IL-1A (−889) allele 2, allele 4 of the gaat.p33330 marker of the IL-1B/IL-1RN intergenic region, allele 6 of the Y31 marker of the IL-1B/IL-1RN intergenic region, IL-1RN (+2018) allele 1, IL-1A (+4845) allele 2, IL-1B (−3737) allele 1, IL-1B (+3954) allele 2 and IL-1RN (VNTR) allele 1.

In yet another embodiment, preferred alleles of the pattern 2 haplotype include: the IL-1A (222/223) allele 4, IL-1A (gz5/gz6) allele 4, IL-1A (−889) allele 1, allele 3 of the gaat.p33330 marker of the IL-1B/IL-1RN intergenic region, allele 3 of the Y31 marker of the IL-1B/IL-1RN intergenic region, IL-1RN (+2018) allele 2, IL-1A (+4845) allele 1, IL-1B (−3737) allele 1, IL-1B (+3954) allele 1 and IL-1RN (VNTR) allele 2.

In a further embodiment, preferred alleles of the pattern 3 haplotype include: the IL-1A (−889) allele 1, IL-1A (+4845) allele 1, IL-1B (−3737) allele 2, IL-1B (+3954) allele 1, IL-1RN (+2018) allele 1, and IL-1RN (VNTR) allele 1.

In particularly preferred embodiments, at least one allele to be detected is selected from the group consisting of IL-1RN (VNTR) allele 2, IL-1RN (+2018) allele 2, IL-1A (+4845) allele 2 and IL-1B (+3954) allele 2.

In another aspect, the invention provides methods for identifying a biomarker that is useful for determining an aging-related phenotype, comprising measuring a biomarker in at least one subject having an EOA-associated allele, and measuring said biomarker in at least one subject not having an EOA-associated allele. A biomarker that shows a substantial difference between subjects with and without an EOA-associated allele is a biomarker that will be useful in monitoring and predicting EOA-related events. In another embodiment, a biomarker may be identified by measuring the biomarker in cell cultures having or not having an EOA-associated allele.

In an additional aspect, the invention provides methods for screening test substances to identify a test substance that is likely to prevent or diminish the early onset of an aging-related condition. Methods of the invention comprise contacting a cell containing DNA comprised of at least one allele of the pattern 1 or pattern 2 haplotypes with a test substance; and observing at least one biomarker in said subject, wherein a change in a biomarker from a senescence-related phenotype to a non-senescence-related phenotype identifies a test substance that is likely to prevent or diminish the early onset of aging-related diseases and conditions.

In a further aspect, the invention provides a method for screening genes to identify a gene that is likely to prevent or diminish the early onset of an aging-related condition in a subject, said method comprising contacting a cell containing DNA comprised of at least one allele of the pattern 1 or pattern 2 haplotypes with a test gene under conditions causing the test gene to enter one or more of said cells; and observing at least one biomarker in said subject, wherein a change in a biomarker from a senescence-related phenotype to a non-senescence-related phenotype identifies a test gene that is likely to prevent or diminish the early onset of aging-related diseases and conditions.

In yet another aspect, the invention provides methods of preventing or diminishing the early onset of an aging-related condition. In one embodiment, a subject is contacted with a substance or gene identified according to the methods described above. In a further aspect, the invention provides methods for preventing or diminishing senescence of cultured cells.

In another aspect, the invention provides methods for determining the stage of an aging-related condition in a subject. The methods comprise observing at least one biomarker identified according to the methods described above and determining the degree to which the biomarker evinces an aging-related phenotype. The greater the degree to which the biomarker evinces an aging related-phenotype, the later the stage of the aging-related condition.

### BRIEF DESCRIPTION OF THE FIGURES

FIG. 1 shows the percentage of subjects, sorted by age, that carry certain IL-1 alleles.

FIG. 2 is a graph showing human umbilical cord vascular endothelial cell (HUVEC) population doublings as a function of IL-1ra genotype (VNTR polymorphism).

FIG. 3 is a graph showing HUVEC population doublings in cells with an IL-1ra (VNTR) 1.1 genotype versus cells with 1.2 or 2.2 genotypes. The decrease in cumulative population doublings is significant at p=0.047.

FIG. 4 shows the nucleic acid sequence for IL-1A (GEN X03833; SEQ ID No. 1).

FIG. 5 shows the nucleic acid sequence for IL-1B (GEN X04500; SEQ ID No. 2).

FIG. 6 shows the nucleic acid sequence for the secreted IL-1RN (GEN X64532; SEQ ID No. 3).

FIG. 7 shows the nucleic acid sequence for the intracellular IL-1RN (GEN X77090; SEQ ID No. 4).

FIG. 8 shows distribution of IL-1 genotypes by age and by age in deciles.

### DETAILED DESCRIPTION

Interleukin-1 and Aging

The invention is based, in part, on the finding that an individual's IL-1 genotype influences the genetic and cellular aspects of aging in that individual. For example, IL-1 alleles are associated with early onset of aging-related conditions. Furthermore, IL-1 alleles are associated with earlier senescence in cultured cells.

It is well established that interleukin-1 expression and activity are associated with aging, both in the whole organism and in isolated cell cultures. However, the relationship between aging and IL-1 is complex, with varying effects in different cell types and tissues. In general, IL-1 production is thought to increase in aging tissues. For example, the urine levels of IL-1β were significantly increased in elderly subjects, indicating a higher IL-1β level in the bloodstream (Liao et al. (1993) Gerontology 39: 19-27). However, in certain tissues, lowered IL-1 production is correlated with aging and may result in different aging-related conditions. IL-1 is thought to affect many aging-related conditions, including but not limited to impaired connective tissue function, cardiovascular disease, age-related cancer, abnormal immune system function and impaired neurological function. IL-1 is also thought to influence conditions that result, at least in part, from the accumulation of calcified products and amyloid, oxidative damage or increased production of reactive oxygen species. In certain aspects, methods of the invention may be used to predict the likelihood of an early onset of any of these conditions. In certain embodiments, the invention relates to the observation that a subject population having a certain IL-1 genotype will, on average, experience an earlier onset of many age-related disorders and, in certain instances, will experience a more rapid progression of age-related disorders. In other aspects, a subject's IL-1 genotype may be used to identify subjects that would be candidates for preventative therapy or an aggressive or early therapy.

Cardiovascular Disease

Cardiovascular disease includes a wide array of conditions, many of which result from the process of atherosclerosis. Aging is an important risk factor for cardiovascular disease, and particulraly ischemic heart disease. Between the ages of 40 and 80, the risk of death from ischemic heart disease rises 100 fold (1996. Mortality Statistic for England and Wales. The Stationary Office, Series DH2 number 23, ISBN 0 11 621025 7). In general there is also an age-related increase in the area of major blood vessels covered by raised lesions of atherosclerosis (White et al. (1950) Circulation 1:645; Strong et al. (1976) Atherosclerosis 23:451-76). Premature aging syndromes are also associated with atherosclerosis.

Cellular senescence may be a pivotal link between the aging process and cardiovascular disease. In one widely accepted hypothesis, atherosclerosis is thought to result from injury to the vascular endothelium. In the healing response to such injury, vascular endothelial cells undergo cell division. The net result is cellular turnover, which may tax the finite mitotic life span of endothelial cells, leading to the formation of abnormally functioning, senescent endothelial cells which are vulnerable to atherogenesis. In vitro, endothelial cells have a finite lifespan that is shorter than that for fibroblasts. During this process, there is an accumulation of markers of senescence (Maciag et al. (1981) J. Cell Biol. 91: 420-6; Thornton et al. (1983) Science 223:623-5). Most human umbilical vein endothelial cells (HUVECs) die of apoptosis rather than entering a state of growth arrest. In vivo, endothelium turns over at a slow rate (Schwartz et al. (1973) Lab. Invest. 28:699-707), but turnover is higher at sites where atheroma development is most likely, such as vessel branch points (Payling et al. (1968) Nature 220:78-9). Endothelial cell turnover is associated with and links many of the best known risk factors for cardiovascular disease. The first detectable event in high fat diet animal models of atherosclerosis is an increase in endothelial cell turnover (Walker et al. (1986) Am. J Pathol. 125:450-9). Increased endothelial cell turnover is an early event in animal models of hypertension and is associated with cigarette smoking (Owens et al. (1985) Circ. Res. 57: 695-705; Pittilo et al. (1982) Thromb. Haemost. 48:173-176). In addition, these risk factors are additive, which suggests that they may share a common mechanism such as cellular turnover.

Telomere length is increasingly accepted as a marker of cellular mitotic age. Telomere length measurements support the hypothesis that cellular senescence is an important process in cardiovascular disease. Telomere restriction fragment (TLF) lengths decline with cellular divisions in cultured endothelial cells, and TLF lengths decline faster in vessels characterized by the development of atherosclerosis (Chang et al. (1995) PNAS USA 92:11190-94). It may be possible to reverse this process by transfecting vascular endothelial cells with telomerase (Bodnar et al. (1998) Science 279:349-52).

In addition, inflammation is now generally regarded as an important component of the pathogenic process of atherosclerosis (Munro, Lab Invest., 58:249-261 (1988); Badimon, et al., Circulation, 87:3-16 (1993); Liuzzo, et al., N.E.J.M., 331(7):417-24 (1994); Alexander, N.E.J.M., 331(7):468-9 (1994)). Damage to endothelial cells that line the vessels leads to an accumulation of inflammatory cytokines, including IL-1, TNFα, and the release of prostanoids and growth factors such as prostaglandin 12 (PGI2), platelet-derived growth factor (PDGF), basic fibroblast growth factor (bFGF), and granulocyte-monocyte cell stimulating factor (GM-CSF). These factors lead to accumulation and regulation of inflammatory cells, such as monocytes, that accumulate within the vessel walls. The monocytes then release additional inflammatory mediators, including IL-1, TNFα, prostaglandin E2, (PGE2), bFGF, and transforming growth factors α and β (TGFα, TGFβ). All of these inflammatory mediators recruit more inflammatory cells to the damaged area, regulate the behavior of endothelial and smooth muscle cells and lead to the accumulation of atheromatous plaques. IL-1 genotype patterns 1, 2 and 3 are all associated with a predisposition to different aspects of cardiovascular disease, including restenosis after angioplasty (U.S. Pat. No. 6,210,877; U.S. patent application Ser. No. 09/320,395, filed May 26, 1999 and U.S. patent application Ser. No. 09/431,352, filed Nov. 11, 1999.)

Several inflammatory products, including IL-1β, have been identified in atherosclerotic lesions or in the endothelium of diseased coronary arteries (Galea, et al., Ath. Thromb. Vasc. Biol., 16:1000-6 (1996)). Also, serum concentrations of IL-1β have been found to be elevated in patients with coronary disease (Hasdai, et al., Heart, 76:24-8 (1996)). Although it was historically believed that the presence of inflammatory agents was responsive to injury or monocyte activation, it is also possible that an abnormal inflammatory response may be causative of coronary artery disease or create an increased susceptibility to the disease.

In addition, it is possible that IL-1 affects cardiovascular disease through an effect on cellular senescence. It has been demonstrated that endothelium aged in culture accumulates IL-1α mRNA and it has been shown that antisense oligonucleotides to IL-1α may extend endothelial cell life in culture (Maier et al. (1990) Science 249: 1570-4). This effect is variable between cultures but remains a strong suggestion of an IL-1 family effect (Garfinkel et al. (1994) PNAS 91:1559-63). In addition, it is well known that IL-1α is growth inhibitory to endothelial cells and blocks growth in G1 phase (Cozzolino et al. (1990) PNAS 87:6487-91). Unpublished work from our own group has shown that as HUVEC age in culture, IL-1β and to a lesser extent IL-1α synthesis is increased.

Neurologicalfunction

Increased neuronal expression of IL-1β is a hallmark of age-related neurodegeneration (Campbell et al. (1998) Neurobiol. Aging 19: 575-9; Murray et al. (1999) Gerontology 45: 136-42). IL-1 expression is greatly increased in the CNS of patients with Alzheimer's disease (Griffin et al. (1989) PNAS 86:7611-15). Furthermore, IL-1 increases expression of amyloid precursor protein (APP), which forms the plaques that typify, and perhaps cause, Alzheimer's disease. In support of this point, Kolsch et al., (2001) Ann Neurol 25:29-41, demonstrated that subjects homozygous for the IL-1A (−889) allele 2 showed a more rapid development of Alzheimer's disease at a population level, as compared to those with one or more IL-1A (−889) allele 1 (consistent with a pattern 1 association, disclosed herein). Recombinant IL-1β inhibits long-term potentiation in the mossy fiber CA3 pathway of the mouse hippocampus (Katsuki et al. (1990) Eur. J. Pharmacol. 181: 323-6). Memory formation is thought to be mediated by LTP in these neurons in the hippocampus. Thus, elevated IL-1β levels may lead to impaired memory (Lynch MA (1998) Prog. Neurobiol. 56:571-89). Furthermore, neuronal function in general depends upon the precise control of fluxes across the cell membrane. IL-1 is known to increase the production of active oxygen species that can oxidize lipids and lead to decreased membrane fluidity. In this manner, IL-1 may damage or unbalance a wide array of neuronal activities (Lynch MA (1998) Prog. Neurobiol. 56:571-89; Murray et al. (1997) Gerontology 45:136-42). IL-1 further acts to stimulate the production of many stress hormones by the hypothalamic-pituitary axis. These stress hormones include ACTH and corticosterone. In aging rats, the regulation of these hormones by IL-1 is blunted, but the stress hormones are produced for a longer period of time. Such altered stress hormone production may play a role in age-related depression, memory loss and other disorders (Scapagnini U (1992) Psychoneuroendocrinology 17:411-420).

Connective Tissue Function

Connective tissues include bone, skin, cartilage, tendons, muscles and ligaments. Many of these tissues exist in a dynamic state, depending on a balance of construction and destruction. In bone, destruction is mediated by osteoclasts. Osteoclast proliferation is stimulated by IL-1 (Mundy et al. (1974) N. Engl. J. Med 290: 867-70). Furthermore, certain IL-1 genotypes are associated with an increased likelihood of osteoporosis (U.S. Pat. No. 5,698,399). Muscle tissue, when placed under stress, develops a focus of inflammatory activity and results in the production of IL-1β. IL-1β accumulates in muscle after exercise-induced stress and can cause breakdown of muscle protein (Cannon et al. (1991) Am. J Physiol. 260:R1214-19; Fielding et al. (1993) Am. J. Physiol. 265:R166-72). Notably, exercise causes greater muscle protein turnover in elderly men than in younger men (Fielding et al. (1997) Int. J. Sports Med. 18:S22-27).

Osteoarthritis and rheumatoid arthritis occur predominantly in middle-aged or older individuals. These diseases result from chronic inflammation of joints and destruction of cartilage. Cartilage is comprised of proteoglycans. IL-1 stimulates proteoglycan breakdown and inhibits proteoglycan synthesis (van Beuningen et al. (1991) Arthritis and Rheumatism 34:606-615). Therefore, increased levels of IL-1 in older individuals may lead to greater destruction of cartilage.

Microarray analysis of fibroblasts from aged and young individuals showed that the expression of genes involved in collagen and connective tissue formation was altered. In addition, genes coding for proteins involved in the inflammatory response (mediated in part by IL-1) were increased (Marx (2000) Science 5462:2390; Ly et al. (2000) Science 287: 2486-92).

IL-6 is increased in older individuals. IL-6 is known to promote bone resorption and be associated with osteoporosis. IL-6 production can be stimulated by IL-1 acting through the NF-κB transcription factor. IL-1 may also promote IL-6 production through other, as yet unknown, pathways (Ershler et al. (2000) Annu. Rev. Med. 51:245-70). Increased IL-6 may also be partly responsible for frailty, anemia, thrombocytosis and dementia.

Age-Related Cancer

Many cancers are strongly age-related (Newell et al. (1989) Semin. Oncol. 16:3-9; Miller (1991) Cancer 68:2496-2501). In fact, incidence of most important cancers increases dramatically with age, approximately as a fourth power of age (Miller (1991) Cancer 11:2496-2501). This age relationship may, in part, be caused by decreasing immune function and the perturbation of normal cellular interactions that prevent excessive proliferation. IL-1α production may have a role in age-related endometrial cancer. Endometrial stromal fibroblasts (ESFs) normally inhibit anchorage-independent proliferation of endometrial cancers, but ESFs from aged individuals actually promote proliferation of cancer cells. Intriguingly, aged ESFs produce increased levels of IL-1α, and antagonizing the IL-1 pathway restores the youth-associated, anti-cancer phenotype. Breast and prostate cancer are also strongly age-related and are influenced by similar interactions with stromal cells (Rinehart et al. (1999) Exp. Cell Res. 248: 599-607). Other cancers with strong increases in incidence with age include cancers of the colon, esophagus, stomach, rectum, pancreas, and nonmelanoma skin cancer.

Immune System Function

It is well known that aging humans and animals show impairments in a variety of immune functions and particularly T cell mediated functions. Immune function in the epidermis is mediated in large part by Langerhans cells (the major antigen presenting cell of the epidermis), and keratinocytes (producers of cytokines, including IL-1). IL-1 production in these cells decreases with age in mice, suggesting that the immune function of the epidermis may become compromised (Sauder et al. (1989) Immunol. Lett. 20:111-4). Aged mice show decreased production of IL-2 by helper T cells. This is thought to contribute to the impairment of helper T cell-mediated immune functions in older mice. This decrease may be caused in part, by the inability of macrophages from aged mice to produce sufficient quantities of IL-1 upon stimulation (Bruley-Rosset et al. (1984) Mech. Aging and Develop. 24:247-64; Inamizu et al. (1985) Immunology 55:447-55).

Amyloid and Calcification

Many diseases of aging individuals result from accumulations of amyloid plaque or calcified tissues. IL-1 is thought to affect these conditions. Alzheimer's disease is thought to be caused, in part, by the accumulation of amyloid plaques. These plaques contain the amyloid protein, which is itself a proteolytic byproduct of the amyloid precursor protein (APP). IL-1 promotes production of APP (Blume et al. (1989) Neurobiol Aging 10:406-8). See Neurological Disorders for other information regarding Alzheimer's disease.

Active Oxygen Species

Oxidative damage has been implicated as a cause of many aging-related conditions. Reactive oxygen species (ROS) can damage proteins, lipids and nucleic acids, leading to the accumulation of mutations as well as damaged cell components, any of which may contribute to the aging process. IL-1 is known to promote the inflammatory process which results in the production of reactive oxygen species. Immune cells such as macrophages and neutrophils produce specialized enzyme systems, such as a form of NADPH oxidase, that produce superoxides. Accordingly, it is hypothesized that IL-1 activity may promote aging, in part, by causing increased production of reactive oxygen species.

At the cellular level, ROS are produced and consumed by a variety of exogenous and endogenous systems. Primary endogenous sources include mitochondria, peroxisomes, lipoxygenases, NADPH oxidases and cytochrome P450s. Primary exogenous sources include ultraviolet light, ionizing radiation, chemotherapeutics, environmental toxins and immune system activities. Antioxidant defenses include catalase, superoxide dismutase, glutathione peroxidase, glutathiones, vitamins (such as A, C and E), etc. A variety of signaling pathways are affected by ROS, including ERK, JNK, p38 MAPK, PI(3)K/AKT, NF-KB, p53 and the heat shock response. In general, ERK, PI(3)K/AKT, NF-κB and the heat shock response contribute to survival in the face of oxidative stress, while JNK, p38 MAPK, and p53 are more commonly associated with apoptosis.

Cellular Effects

In addition to the in vivo IL-1 effects on aging, described above, IL-1 has an effect on the senescence of cells in culture. It is postulated that the effects of IL-1 on senescence of cultured cells mirror the effects of IL-1 on cells in vivo and may underlie most or all IL-1-mediated effects on aging processes in vivo. IL-1a and the expression of IL-1 oinducible genes increase with age. Senescent human endothelial cells express increased levels of IL-1α and nuclear localization of IL-1α correlates with impaired cell growth. IL-1 stimulates the production of ether-linked diglyceride species (alkyl, acyl and alkenyl and acylglycerols) which have been found to inhibit calcium-insensitive protein kinase C isotypes. These PKC isotypes are linked to mitogenic activity; therefore inhibition of these isotypes via IL-1-stimulated diglyceride production may play a role in growth arrest of cells.

In addition, cellular senescence may result from premature apoptosis. Apoptosis is a programmed cell death and is promoted, in some cases, by the NF-kB transcription factor. NF-kB is stimulated by IL-1 activity and, accordingly, IL-1 activity may promote apoptosis in some circumstances. This may represent yet another mechanism by which IL-1 activity affects cellular senescence.

In addition, as noted above, IL-1 affects telomere maintenace and cellular lifespan. IL-1 signaling may affect telomere maintenance through an effect on mortalin. IL-1 type 1 receptor (IL-1RI) is associated intracellularly with mortalin, a member of the HSP70 family that is associated with cellular mortal phenotype. Cytosolic mortalin is associated with suppression of cellular telomeric maintenance mechanisms. The functional implications of this association are not clear however the association between IL-1RI and mortalin may be one of the pathways by which IL-1 may play a role in replicative senescence.

These studies and others indicate that the genes of the IL-1 locus have a complex pattern of expression and effects in aging organisms. It is likely that these factors affect the aging process through several distinct mechanisms in different tissues.

Angiogenesis

It is anticipated that IL-1 may play a role in angiogenesis. As disclosed herein, the presence of certain IL-1 alleles is indicative of a decreased potential for cell division in endothelial cells. Such decreased potential may impact a wide array of conditions and disorders. For example, the process of angiogenesis requires cell division of endothelial cells to form a tube that becomes a new blood vessel. Angiogenesis occurs in a range of disease states, tumor metastases and abnormal growths by endothelial cells. The vasculature created as a result of angiogenic processes supports the pathological damage seen in these conditions. The diverse pathological states created due to unregulated angiogenesis have been grouped together as angiogenic dependent or angiogenic associated diseases. In addition, angiogenesis is a part of normal processes such as wound healing and reproduction.

Definitions

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “an aberrant activity”, as applied to an activity of a polypeptide such as IL-1, refers to an activity which differs from the activity of the wild-type or native polypeptide or which differs from the activity of the polypeptide in a healthy subject. An activity of a polypeptide can be aberrant because it is stronger than the activity of its native counterpart. Alternatively, an activity can be aberrant because it is weaker or absent relative to the activity of its native counterpart. An aberrant activity can also be a change in an activity. For example an aberrant polypeptide can interact with a different target peptide. A cell can have an aberrant IL-1 activity due to overexpression or underexpression of an IL-1 locus gene encoding an IL-1 locus polypeptide.

“Age-related cancer” refers to any form of neoplasm that occurs with increased incidence in older populations. Most cancers are age-related, and cancers such as breast, prostate and endometrial cancers are particularly so.

“Aging-related conditions” are any of the spectrum of health conditions that occur with increasing frequency in older populations. Such health conditions include, without limitation, impaired connective tissue function (e.g. osteoporosis, osteoarthritis, decreased chondrocyte proteoglycan synthesis and rheumatoid arthritis, decreased wound healing, frailty, muscle wasting), abnormal immune system function (e.g. reduced T cell proliferation, increased IL-1 production, decreased responsiveness to IL-1, increased susceptibility to infectious disease), impaired neurological function (e.g. Alzheimer's disease, dementia, impaired long-term and, impaired hearing, impaired eye function, including retinal degeneration, short-term memory, decreased synaptic plasticity, impaired learning, insomnia, depression), cardiovascular disease (e.g. coronary artery disease, stroke, peripheral artery disease, atherosclerosis, congestive heart failure, hypertension) and age-related cancers.

An “Aging-associated phenotype” is a phenotype of subjects or cells that is associated with EOA or associated with an increased likelihood of EOA. An EOA-associated phenotype is also any phenotype found in a subject or cell having an EOA-associated allele, where such phenotype differs from that found in subjects or cells lacking an EOA-associated allele. Such phenotypes encompass essentially any characteristic of a biomarker. An EOA-associated phenotype may not be directly involved in EOA but may nonetheless serve as an indicator for EOA. A “non-EOA-associated phenotype” is a phenotype that is not associated with EOA or with an increased likelihood of developing EOA.

The term “allele” refers to the different sequence variants found at different polymorphic regions. For example, IL-1RN (VNTR) has at least five different alleles. The sequence variants may be single or multiple base changes, including without limitation insertions, deletions, or substitutions, or may be a variable number of sequence repeats.

The term “allelic pattern” refers to the identity of an allele or alleles at one or more polymorphic regions. For example, an allelic pattern may consist of a single allele at a polymorphic site, as for IL-1RN (VNTR) allele 1, which is an allelic pattern having at least one copy of IL-1 RN allele 1 at the VNTR of the IL-1 RN gene loci. Alternatively, an allelic pattern may consist of either a homozygous or heterozygous state at a single polymorphic site. For example, IL1-RN (VNTR) allele 2,2 is an allelic pattern in which there are two copies of the second allele at the VNTR marker of IL-1RN and that corresponds to the homozygous IL-RN (VNTR) allele 2 state. Alternatively, an allelic pattern may consist of the identity of alleles at more than one polymorphic site.

The term “antibody” as used herein is intended to refer to a binding agent including a whole antibody or a binding fragment thereof which is specifically reactive with an IL-1B polypeptide. Antibodies can be fragmented using conventional techniques and the fragments screened for utility in the same manner as described above for whole antibodies. For example, F(ab)₂ fragments can be generated by treating an antibody with pepsin. The resulting F(ab)₂ fragment can be treated to reduce disulfide bridges to produce Fab fragments. The antibody of the present invention is further intended to include bispecific, single-chain, and chimeric and humanized molecules having affinity for an IL-1B polypeptide conferred by at least one CDR region of the antibody.

“Biological activity” or “bioactivity” or “activity” or “biological function”, which are used interchangeably, for the purposes herein means an effector or antigenic function that is directly or indirectly performed by an IL-1 polypeptide (whether in its native or denatured conformation), or by any subsequence thereof. These terms are also intended to encompass properties of IL-1 proteins and genes, such as expression levels and post-translational modifications. Biological activities include binding to a target peptide, e.g., an IL-1 receptor. An IL-1 bioactivity can be modulated by directly affecting an IL-1 polypeptide. Alternatively, an IL-1 bioactivity can be modulated by modulating the level of an IL-1 polypeptide, such as by modulating expression of an IL-1 gene.

As used herein the term “bioactive fragment of an IL-1 polypeptide” refers to a fragment of a full-length IL-1 polypeptide, wherein the fragment specifically mimics or antagonizes the activity of a wild-type IL-1 polypeptide. The bioactive fragment preferably is a fragment capable of interacting with an interleukin receptor.

The term “biomarker” refers to a phenotype of a subject or cells. Biomarkers encompass a broad range of intra- and extra-cellular events as well as whole organism physiological changes. Biomarkers may be any of these and are not necessarily involved in inflammatory responses. With respect to cells, biomarkers may be essentially any aspect of cell function, for example: levels or rate of production of signaling molecules, transcription factors, intermediate metabolites, cytokines, prostanoids, steroid hormones (e.g. estrogen, progesterone, androstenedione or testosterone), gonadotropins (e.g. LH and FSH), gene transcripts, post-translational modifications of proteins, gonadotropin releasing hormone (GnRH), catecholamines (e.g. dopamine or norepinephrine), opioids, activin, inhibin, as well as IL-1 bioactivities. Biomarkers may include whole genome analysis of transcript levels or whole proteome analysis of protein levels and/or modifications. Additionally, biomarkers may be reporter genes. For example, an IL-1 promoter or an IL-1 promoter comprising an EOA-associated allele can be operationally linked to a reporter gene. In an alternative method, the promoter can be an IL-1-regulated promoter, such as IL-8. In this manner, the activity of the reporter gene is reflective of the activity of the promoter. Suitable reporter genes include GUS, LacZ, green fluorescent protein (GFP) (and variants thereof, such as Red Fluorescent Protein, Cyan Fluorescent Protein, Yellow Fluorescent Protein and Blue Fluorescent Protein), or essentially any other gene whose product is easily detected. Other preferred biomarkers include factors involved in immune and inflammatory responses, as well as factors involved in IL-1 production and signaling, as described below. In subjects, biomarkers can be, for example, any of the above as well as electrocardiogram parameters, pulmonary function, IL-6 activities, urine parameters or tissue parameters. “EOA associated biomarkers” are any of the above which are found to correlate with EOA, or which are preferentially found in subjects or cells comprising an EOA-associated allele.

A “cardiovascular disease” is a cardiovascular disorder, as defined herein, characterized by clinical events including clinical symptoms and clinical signs. Clinical symptoms are those experiences reported by a patient that indicate to the clinician the presence of pathology. Clinical signs are those objective findings on physical or laboratory examination that indicate to the clinician the presence of pathology. “Cardiovascular disease” includes both “coronary artery disease” and “peripheral vascular disease” (which includes cerebrovascular disease). Clinical symptoms in cardiovascular disease include chest pain, shortness of breath, weakness, fainting spells, alterations in consciousness, extremity pain, paroxysmal nocturnal dyspnea, orthophea, transient ischemic attacks and other such phenomena experienced by the patient. Clinical signs in cardiovascular disease include such findings as EKG abnormalities, altered peripheral pulses, arterial bruits, abnormal heart sounds, rates, jugular venous distention, neurological alterations and other such findings discerned by the clinician. Clinical symptoms and clinical signs can combine in a cardiovascular disease such as a myocardial infarction (MI) or a stroke (also termed a “cerebrovascular accident” or “CVA”), where the patient will report certain phenomena (symptoms) and the clinician will perceive other phenomena (signs) all indicative of an underlying pathology. “Cardiovascular disease” includes those diseases related to the cardiovascular disorders of fragile plaque disorder, occlusive disorder and stenosis. For example, a cardiovascular disease resulting from a fragile plaque disorder, as that term is defined below, can be termed a “fragile plaque disease.” Clinical events associated with fragile plaque disease include those signs and symptoms where the rupture of a fragile plaque with subsequent acute thrombosis or with distal embolization are hallmarks. Examples of fragile plaque disease include certain strokes and myocardial infarctions. As another example, a cardiovascular disease resulting from an occlusive disorder can be termed an “occlusive disease.” Clinical events associated with occlusive disease include those signs and symptoms where the progressive occlusion of an artery affects the amount of circulation that reaches a target tissue. Progressive arterial occlusion may result in progressive ischemia that may ultimately progress to tissue death if the amount of circulation is insufficient to maintain the tissues. Signs and symptoms of occlusive disease include claudication, rest pain, angina, and gangrene, as well as physical and laboratory findings indicative of vessel stenosis and decreased distal perfusion. As yet another example, a cardiovascular disease resulting from restenosis can be termed an in-stent stenosis disease. In-stent stenosis disease includes the signs and symptoms resulting from the progressive blockage of an arterial stent that has been positioned as part of a procedure such as, for example, percutaneous transluminal angioplasty, where the presence of the stent is intended to help hold the vessel in its newly expanded configuration. The clinical events that accompany in-stent stenosis disease are those attributable to the restenosis of the reconstructed artery.

A “cardiovascular disorder” refers broadly to both to coronary artery disorders and peripheral arterial disorders (including cerebrovascular disorders). The term “cardiovascular disorder” can apply to any abnormality of an artery, whether structural, histological, biochemical or any other abnormality. This term includes those disorders characterized by fragile plaque (termed herein “fragile plaque disorders”), those disorders characterized by vaso-occlusion (termed herein “occlusive disorders”), and those disorders characterized by restenosis. A “cardiovascular disorder” can occur in an artery primarily, that is, prior to any medical or surgical intervention. Primary cardiovascular disorders include, among others, atherosclerosis, arterial occlusion, aneurysm formation and thrombosis. A “cardiovascular disorder” can occur in an artery secondarily, that is, following a medical or surgical intervention. Secondary cardiovascular disorders include, among others, post-traumatic aneurysm formation, restenosis, and post-operative graft occlusion.

“Cells”, “host cells” or “recombinant host cells” are terms used interchangeably herein to refer not only to the particular subject cell, but to the progeny or potential progeny of such a cell. Because certain modifications may occur in succeeding generations due to either mutation or environmental influences, such progeny may not, in fact be identical to the parent cell, but is still included within the scope of the term as used herein.

A “chimera,” “mosaic,” “chimeric mammal” and the like, refers to a transgenic mammal with a knock-out or knock-in construct in at least some of its genome-containing cells.

The terms “comprise” and “comprising” is used in the inclusive, open sense, meaning that additional elements may be included.

The terms “control” or “control sample” refer to any sample appropriate to the detection technique employed. The control sample may contain the products of the allele detection technique employed or the material to be tested. Further, the controls may be positive or negative controls. By way of example, where the allele detection technique is PCR amplification, followed by size fractionation, the control sample may comprise DNA fragments of an appropriate size. Likewise, where the allele detection technique involves detection of a mutated protein, the control sample may comprise a sample of a mutant protein. However, it is preferred that the control sample comprises the material to be tested. For example, the controls may be a sample of genomic DNA or a cloned portion of the IL-1 gene cluster. However, where the sample to be tested is genomic DNA, the control sample is preferably a highly purified sample of genomic DNA.

A “clinical event” is an occurrence of clinically discernible signs of a disease or of clinically reportable symptoms of a disease. “Clinically discernible” indicates that the sign can be appreciated by a health care provider. “Clinically reportable” indicates that the symptom is the type of phenomenon that can be described to a health care provider. A clinical event may comprise clinically reportable symptoms even if the particular patient cannot himself or herself report them, as long as these are the types of phenomena that are generally capable of description by a patient to a health care provider.

“Connective tissue” is a well known term and refers to tissues including bone, muscle, cartilage, tendons, ligaments and skin. Connective tissue functions include structural integrity, flexibility, deformability, tensile strength, contractile strength, ability to heal, remodel and resist damage.

A “disorder associated allele” or “an allele associated with a disorder” refers to an allele whose presence in a subject indicates that the subject has or is susceptible to developing a particular disorder. One type of disorder associated allele is a “senescence associated allele,” the presence of which in a subject indicates that the subject is susceptible to premature senescence or premature onset of aging related disorders. These include alleles associated with decreased capacity for cell division, cellular senescence and earlier death. Examples of senescence associated alleles include those alleles comprising the IL-1 pattern 1—i.e. allele 2 of the IL-1A +4825; allele 2 of the +3954 marker of IL-1B; and allele 1 of the +2018 marker of IL-1RN; and allele 1 of the (−511) marker of the IL-1 B gene or an allele that is in linkage disequilibrium with one of the aforementioned alleles.

The phrases “disruption of the gene” and “targeted disruption” or any similar phrase refers to the site specific interruption of a native DNA sequence so as to prevent expression of that gene in the cell as compared to the wild-type copy of the gene. The interruption may be caused by deletions, insertions or modifications to the gene, or any combination thereof.

“Early onset of aging related conditions” or or “early onset or progression of aging related conditions” or “EOA” refers to a situation wherein an aging-related condition occurs earlier or progresses earlier than would otherwise have been expected for the particular individual and the particular condition. The expected age of onset may vary depending on the amount of information known about that individual. For example, many conditions affect women at a later date than men, and therefore, for those conditions, the expected age of onset would be later for women. An individual with a Werner's syndrome mutation would have an expected onset of aging related conditions at approximately 10 to 15 years of age, far earlier than the population as a whole. In the absence of any information about an individual, the expected onset age would be that for the population as a whole for the condition in question.

An “EOA therapeutic” refers to any agent that prevents or postpones the development or alleviates the symptoms of early onset of aging-related conditions. An EOA therapeutic can be a polypeptide, peptidomimetic, nucleic acid, other inorganic or organic molecule, or a nutraceutical, preferably a “small molecule”. Preferably an EOA therapeutic can modulate at least one EOA-associated phenotype. For example, an EOA therapeutic may modulate an activity of an IL-1 polypeptide, e.g., interaction with an IL-1 receptor, by mimicking or potentiating (agonizing) or inhibiting (antagonizing) the effects of a naturally-occurring IL-1 polypeptide. An IL-1 agonist can be a wild-type IL-1 protein or derivative thereof having at least one bioactivity of the wild-type IL-1, e.g. receptor binding activity. An IL-1 agonist can also be a compound that upregulates expression of an IL-1 gene or which increases at least one bioactivity of an IL-1 protein. An agonist can also be a compound which increases the interaction of an IL-1 polypeptide with another molecule, e.g., an interleukin receptor. An IL-1 antagonist can be a compound which inhibits or decreases the interaction between an IL-1 protein and another molecule, e.g., a receptor, such as an IL-1 receptor. Accordingly, a preferred antagonist is a compound which inhibits or decreases binding to an IL-1 receptor and thereby blocks subsequent activation of the IL-1 receptor. An antagonist can also be a compound that downregulates expression of an IL-1 locus gene or which reduces the amount of an IL-1 protein present. The IL-1 antagonist can be a dominant negative form of an IL-1 polypeptide, e.g., a form of an IL-1 polypeptide which is capable of interacting with a target peptide, e.g., an IL-1 receptor, but which does not promote the activation of the IL-1 receptor. The IL-1 antagonist can also be a nucleic acid encoding a dominant negative form of an IL-1 polypeptide, an IL-1 antisense nucleic acid, or a ribozyme capable of interacting specifically with an IL-1 RNA. Yet other IL-1 antagonists are molecules which bind to an IL-1 polypeptide and inhibit its action. Such molecules include peptides, e.g., forms of IL-1 target peptides which do not have biological activity, and which inhibit binding by IL-1 to IL-1 receptors. Thus, such peptides will bind the active site of IL-1 and prevent it from interacting with target peptides, e.g., an IL-1 receptor. Yet other IL-1 antagonists include antibodies interacting specifically with an epitope of an IL-1 molecule, such that binding interferes with the biological function of the IL-1 locus polypeptide. In yet another preferred embodiment, the IL-1 antagonist is a small molecule, such as a molecule capable of inhibiting the interaction between an IL-1 polypeptide and a target IL-1 receptor. Alternatively, the small molecule can function as an antagonist by interacting with sites other than the IL-1 receptor binding site. An antagonist can be any class of molecule, including a nucleic acid, protein, carbohydrate, lipid or combination thereof, but for therapeutic purposes is preferably a small molecule.

The term “early progression of an aging-related condition” or “EPA” is used to indicate a situation wherein the rate at which an aging related condition progresses in a subject is more rapid than in the population as a whole. Early onset and early progression are strongly overlapping and related situations, and, unless clearly indicated by context, each of the embodiments described with respect to early onset may also be applied to early progression.

The term “haplotype” as used herein is intended to refer to a set of alleles that are inherited together as a group (are in linkage disequilibrium) at statistically significant levels (p_{corr}≦0.05). As used herein, the phrase “an IL-1 haplotype” refers to a haplotype in the IL-1 loci. At least three IL-1 proinflammatory haplotypes are known. The IL-1 (44112332) (also referred to herein as pattern 2) haplotype is associated with decreased IL-receptor antagonist activity, whereas the IL-1 (33221461) (also referred to herein as pattern 1) haplotype is associated with increased IL-1α and β agonist activity. The IL-1 (44112332) haplotype includes the following alleles: IL-1RN (+2018) allele 2; IL-1RN (VNTR) allele 2; IL-1A (222/223) allele 4; IL-1A (gz5/gz6) allele 4; IL-1A (−889) allele 1; IL-1B (+3954) allele 1; IL-1B (−3737) allele 1; IL-1B (−511) allele 2; gaat.p33330 allele 3; Y31 allele 3; IL-1RN exon lic (1812) allele 2; IL-1RN exon lic (1868) allele 2; IL-1RN exon lic (1887) allele 2; Pic (1731) allele 2; IL-1A (+4845) allele 1; IL-1B (+6912) allele 1; IL-1B (−31) allele 2. The IL-1 (33221461) haplotype includes the following alleles: IL-1RN (+2018) allele 1; IL-1RN (VNTR) allele 1; IL-1A (222/223) allele 3; IL-1A (gz5/gz6) allele 3; IL-1A (−889) allele 2; IL-1B (+3954) allele 2; IL-1B (−3737) allele 1; IL-1B (−511) allele 1; gaat.p33330 allele 4; Y31 allele 6; IL-1RN exon lic (1812) allele 1; IL-1RN exon lic (1868) allele 1; IL-1RN exon lic (1887) allele 1; Pic (1731) allele 1; IL-1A (+4845) allele 2; IL-1B (+6912) allele 2; IL-1B (−31) allele 1. A third haplotype (pattern 3) comprises the following alleles: IL-1A (+4845) allele 1; IL-1A (−889) allele 1; IL-1B (+3954) allele 1; IL-1B (−511) allele 1; IL-1B (−3737) allele 2; IL-1RN (+2018) allele 1; IL-1RN (VNTR) allele 1.

An “IL-1 agonist” as used herein refers to an agent that mimics, upregulates (potentiates or supplements) or otherwise increases an IL-1 bioactivity or a bioactivity of a gene in an IL-1 biological pathway. IL-1 agonists may act on any of a variety of different levels, including regulation of IL-1 gene expression at the promoter region, regulation of mRNA splicing mechanisms, stabilization of mRNA, phosphorylation of proteins for translation, conversion of proIL-1 to mature IL-1 and secretion of IL-1. Agonists that increase IL-1 synthesis include: lipopolysaccharides, IL-1B, cAMP inducing agents, NFκKB activating agents, AP-1 activating agents, TNF-α, oxidized LDL, advanced glycosylation end products (AGE), sheer stress, hypoxia, hyperoxia, ischemia reperfusion injury, histamine, prostaglandin E 2 (PGE2), IL-2, IL-3, IL-12, granulocyte macrophage-colony stimulating factor (GM-CSF), monocyte colony stimulating factor (M-CSF), stem cell factor, platelet derived growth factor (PDGF), complement C5A, complement C5b9, fibrin degradation products, plasmin, thrombin, 9-hydroxyoctadecaenoic acid, 13-hydroxyoctadecaenoic acid, platelet activating factor (PAF), factor H, retinoic acid, uric acid, calcium pyrophosphate, polynucleosides, c-reactive protein, antitrypsin, tobacco antigen, collagen, integrins, LFA-3, anti-HLA-DR, anti-IgM, anti-CD3, CD40 ligation, phytohemagglutinin (CD2), sCD23, ultraviolet B radiation, gamma radiation, substance P,. isoproterenol, methamphetamine and melatonin. Agonists that stabilize IL-1 mRNA include bacterial endotoxin and IL-1. Other agonists, that function by increasing the number of IL-1 type 1 receptors available, include IL-1, PKC activators, dexamethasone, IL-2, IL-4 and PGE2. Other preferred antagonists interfere or inhibit signal transduction factors activated by IL-1 or utilized in an IL-1 signal transduction pathway (e.g. NFκB and AP-1, P13 kinase, phospholipase A2, protein kinase C, JNK-1, 5-lipoxygenase, cyclooxygenase 2, tyrosine phospborylation, iNOS pathway, Rac, Ras, TRAF). Still other agonists increase the bioactivity of genes whose expression is induced by IL-1, including: IL-1, IL-1Ra, TNF, IL-2, IL-3, IL-6, IL-12, GM-CSF, G-CSF, TGF, fibrinogen, urokinase plasminogen inhibitor, Type 1 and type 2 plasminogen activator inhibitor, p-selectin (CD62), fibrinogen receptor, CD-11/CD18, protease nexin-1, CD44, Matrix metalloproteinase-1 (MMP-1), MMP-3, Elastase, Collagenases, Tissue inhibitor of metalloproteinases-1 (TIMP-1), Collagen, Triglyceride increasing Apo CIII, Apolipoprotein, ICAM-1, ELAM-1, VCAM-1, L-selectin, Decorin, stem cell factor, Leukemia inhibiting factor, IFNa,b,g, L-8, IL-2 receptor, IL-3 receptor, IL-5 receptor, c-kit receptor, GM-CSF receptor, Cyclooxygenase-2 (COX-2), Type 2 phospholipase A2, Inducible nitric oxide synthase (iNOS), Endothelin-1,3, Gamma glutamyl transferase, Mn superoxide dismutase, C-reactive protein, Fibrinogen, Serum amyloid A, Metallothioneins, Ceruloplasmin, Lysozyme, Xanthine dehydrogenase, Xanthine oxidase, Platelet derived growth factor A chain (PDGF), Melanoma growth stimulatory activity (gro-a,b,g), Insulin-like growth factor-i (IGF-1), Activin A, Pro-opiomelanocortiotropin, corticotropin releasing factor, B amyloid precursor, Basement membrane protein-40, Laminin B1 and B2, Constitutive heat shock protein p70, P42 mitogen, activating protein kinase, omithine decarboxylase, heme oxygenase and G-protein a subunit).

An “IL-1 antagonist” as used herein refers to an agent that downregulates or otherwise decreases an IL-1 bioactivity. IL-1 antagonists may act on any of a variety of different levels, including regulation of IL-1 gene expression at the promoter region, regulation of mRNA splicing mechanisms, stabilization of mRNA, phosphorylation of proteins for translation, conversion of proIL-1 to mature IL-1 and secretion of IL-1. Antagonists of IL-1 production include: corticosteroids, lipoxygenase inhibitors, cyclooxygenase inhibitors, γ-interferon, IL-4, IL-10, IL-13, transforming growth factor β (TGF-β), ACE inhibitors, n-3 polyunsaturated fatty acids, antioxidants and lipid reducing agents. Antagonists that destabilize IL-1mRNA include agents that promote deadenylation. Antagonists that inhibit or prevent phosphorylation of IL-1 proteins for translation include pyridinyl-imadazole compounds, such as tebufelone and compounds that inhibit microtubule formation (e.g. colchicine, vinblastine and vincristine). Antagonists that inhibit or prevent the conversion of proIL-1 to mature IL-1 include interleukin converting enzyme (ICE) inhibitors, CXrm-A, transcript X, endogenous tetrapeptide competitive substrate inhibitor, trypsin, elastase, chymotrypsin, chymase, and other nonspecific proteases. Antagonists that prevent or inhibit the scretion of IL-1 include agents that block anion transport. Antagonists that interefere with IL-1 receptor interactions, include: agents that inhibit glycosylation of the type I IL-1 receptor, antisense oligonucleotides against IL-1 RI, antibodies to IL-1RI and antisense oligonucleotides against IL-1RacP. Other antagonists, that function by decreasing the number of IL-1 type 1 receptors available, include TGF-α, COX inhibitors, factors that increase IL-1 type II receptors, dexamethasone, PGE2, IL-1 and IL-4. Other preferred antagonists interfere or inhibit signal transduction factors activated by IL-1 or utilized in an IL-1 signal transduction pathway (e.g NFkB and AP-1, P13 kinase, phospholipase A2, protein kinase C, JNK-1, 5-lipoxygenase, cyclooxygenase 2, tyrosine phosphorylation, iNOS pathway, Rac, Ras, TRAF). Still other antagonists interfere with the bioactivity of genes whose expression is induced by IL-1, including: IL-1, IL-1Ra, TNF, IL-2, IL-3, IL-6, IL-12, GM-CSF, G-CSF, TGF-, fibrinogen, urokinase plasminogen inhibitor, Type 1 and Type 2 plasminogen activator inhibitor, p-selectin (CD62), fibrinogen receptor, CD-11/CD18, protease nexin-1, CD44, Matrix metalloproteinase-1 (MMP-1), MMP-3, Elastase, Collagenases, Tissue inhibitor of metalloproteinases-1 (TIMP-1), Collagen, Triglyceride increasing Apo CIII, Apolipoprotein, ICAM-1, ELAM-1, VCAM-1, L-selectin, Decorin, stem cell factor, Leukemia inhibiting factor, IFN α, β, γ L-8, IL-2 receptor, IL-3 receptor, IL-5 receptor, c-kit receptor, GM-CSF receptor, Cyclooxygenase-2 (COX-2), Type 2 phospholipase A2, Inducible nitric oxide synthase (iNOS), Endothelin-1,3, Gamma glutamyl transferase, Mn superoxide dismutase, C-reactive protein, Fibrinogen, Serum amyloid A, Metallothioneins, Ceruloplasmin, Lysozyme, Xanthine dehydrogenase, Xanthine oxidase, Platelet derived growth factor A chain (PDGF), Melanoma growth stimulatory activity (gro-a,b,g), Insulin-like growth factor-1 (IGF-1), Activin A, Pro-opiomelanocortiotropin, corticotropin releasing factor, B amyloid precursor, Basement membrane protein-40, Laminin B 1 and B2, Constitutive heat shock protein p70, P42 mitogen, activating protein kinase, ornithine decarboxylase, heme oxygenase and G-protein a subunit). Other preferred antagonists include: hymenialdisine, herbimycines (e.g. herbamycin A), CK-103A and its derivatives (e.g. 4,6-dihydropyridazino[4,5-c]pyridazin-5 (1H)-one), CK-119, CK-122, iodomethacin, aflatoxin B1, leptin, heparin, bicyclic imidazoles (e.g SB203580), PD15306 HC1, podocarpic acid derivatives, M-20, Human [Gly2] Glucagon-like peptide-2, FR167653, Steroid derivatives, glucocorticoids, Quercetin, Theophylline, NO-synthetase inhibitors, RWJ 68354, Euclyptol (1.8-cineole), Magnosalin, N-Acetylcysteine, Alpha-Melatonin-Stimulating Hormone (a-MSH), Triclosan (2,4,4′-trichloro-2′-hydroxyldiphenyl ether), Prostaglandin E2 and 4-aminopyridine Ethacrynic acid and 4,4′-diisothiocyanatostilbene-2,2′-disulfonic acid (DIDS), Glucose, Lipophosphoglycan, aspirin, Catabolism-blocking agents, Diacerhein, Thiol-modulating agents, Zinc, Morphine, Leukotriene biosynthesis inhibitors (e.g. MK886), Platelet-activating factor receptor antagonists (e.g. WEB 2086), Amiodarone, Tranilast, S-methyl-L-thiocitrulline, Beta-adrenoreceptor agonists (e.g. Procaterol, Clenbuterol, Fenoterol, Terbutaline, Hyaluronic acid, anti-TNF-α antibodies, anti-IL-1α autoantibodies, IL-1 receptor antagonist, IL-1R-associated kinase, soluble TNF receptors and antiinflammatory cytokines (e.g. IL-4, IL-13, IL-10, IL-6, TGF-β, angiotensin II, Soluble IL-1 type II receptor, Soluble IL-1 type I receptor, Tissue plasminogen activator, Zinc finger protein A20 IL-1 Peptides (e.g. (Thr-Lys-Pro-Arg) (Tuftsin), (Ile-Thr-Gly-Ser-Glu) IL-1-alpha, Val-Thr-Lys-Phe-Tyr-Phe, Val-Thr-Asp-Phe-Tyr-Phe, Interferon alpha2b, Interferon beta, IL-1-beta analogues (e.g. IL-1-beta tripeptide: Lys-D-Pro-Thr), glycosylated IL-1-alpha, and IL-1ra peptides.

“IL-1 gene cluster” and “IL-1 loci” as used herein include all the nucleic acid at or near the 2q13 region of chromosome 2, including at least the IL-1A, IL-1B and IL-1RN genes and any other linked sequences. The terms “IL-1A”, “IL-1B”, and “IL-1RN” as used herein refer to the genes coding for IL-1α, IL-1β, and IL-1 receptor antagonist or IL-1ra, respectively. The DNA in this region has been mapped. Nicklin et al., Genomics 19:382-84, 1994; Nothwang H. G., et al., Genomics 41:370, 1997; Clark, et al., Nucl. Acids. Res. 14:7897-914, 1986, (erratum at Nucleic Acids Res. 15:868, 1987. The gene accession numbers (GEN) for IL-1A and IL-1B, are X03833 and X04500, respectively. In general, references to nucleotide positions for IL-1RN refer to the nucleotide sequence in GEN X64532, which is the secreted form of the protein, unless there is some indication, either expressly indicated or implied from the context, that the intracellular form, which has GEN X77090, is being referenced. The two forms of IL-1RA are encoded by a single gene by alternative use of two first exons. See generally Lennard et al., Crit. Rev. Immuno. 15:77-105, 1995.

“IL-1 functional mutation” refers to a mutation within the IL-1 gene cluster that results in an altered phenotype (i.e. effects the function of an IL-1 gene or protein). Examples include: IL-1A(+4845) allele 2, IL-1B (+3954) allele 2, IL-1B (+6912) allele 2 and IL-1RN (+2018) allele 2.

“IL-1X (Z) allele Y” refers to a particular allelic form, designated Y, occurring at an IL-1 locus polymorphic site in gene X, wherein X is IL-1A, B, or RN or some other gene in the IL-1-gene loci, and positioned at or near nucleotide Z, wherein nucleotide Z is numbered relative to the major transcriptional start site, which is nucleotide +1, of the particular IL-1 gene X. As further used herein, the term “IL-1X allele (Z)” refers to all alleles of an IL-1 polymorphic site in gene X positioned at or near nucleotide Z. For example, the term “IL-1RN (+2018) allele” refers to alternative forms of the IL-1RN gene at marker +2018. “IL-1RN (+2018) allele 1 ” refers to a form of the IL-1RN gene which contains a cytosine (C) at position +2018 of the sense strand. Clay et al., Hum. Genet. 97:723-26, 1996. “IL-1RN (+2018) allele 2” refers to a form of the IL-1RN gene which contains a thymine (T) at position +2018 of the plus strand. When a subject has two identical IL-1RN alleles, the subject is said to be homozygous, or to have the homozygous state. When a subject has two different IL-1 RN alleles, the subject is said to be heterozygous, or to have the heterozygous state. The term “IL-1RN (+2018) allele 2,2” refers to the homozygous IL-1 RN (+2018) allele 2 state. Conversely, the term “IL-1RN (+2018) allele 1,1” refers to the homozygous IL-1 RN (+2018) allele 1 state. The term “IL-1RN (+2018) allele 1,2” refers to the heterozygous allele 1 and 2 state.

“IL-1 related” as used herein is meant to include all genes related to the human IL-1 locus genes on human chromosome 2 (2q 12-14). These include IL-1 genes of the human IL-1 gene cluster located at chromosome 2 (2q 13-14) which include: the IL-1A gene which encodes interleukin-la, the IL-1B gene which encodes interleukin-1β, and the IL-1RN (or IL-1ra) gene which encodes the interleukin-1 receptor antagonist. Furthermore these IL-1 related genes include the type I and type II human IL-1 receptor genes located on human chromosome 2 (2q12) and their mouse homologs located on mouse chromosome 1 at position 19.5 cM. Interleukin-1, interleukin-1, and interleukin-1RN are related in so much as they all bind to IL-1 type I receptors, however only interleukin-1 and interleukin-1 are agonist ligands which activate IL-1 type I receptors, while interleukin-1RN is a naturally occurring antagonist ligand. Where the term “IL-1” is used in reference to a gene product or polypeptide, it is meant to refer to all gene products encoded by the interleukin-1 locus on human chromosome 2 (2q 12-14) and their corresponding homologs from other species or functional variants thereof. The term IL-1 thus includes secreted polypeptides which promote an inflammatory response, such as IL-1 and IL-1β, as well as a secreted polypeptide which antagonize inflammatory responses, such as IL-1α receptor antagonist and the IL-1 type II (decoy) receptor.

An “IL-1 receptor” or “IL-1R” refers to various cell membrane bound protein receptors capable of binding to and/or transducing a signal from IL-1 locus-encoded ligand. The term applies to any of the proteins which are capable of binding interleukin-1 (IL-1) molecules and, in their native configuration as mammalian plasma membrane proteins, presumably play a role in transducing the signal provided by IL-1 to a cell. As used herein, the term includes analogs of native proteins with IL-1-binding or signal transducing activity. Examples include the human and murine IL-1 receptors described in U.S. Pat. No. 4,968,607. The term “IL-1 nucleic acid” refers to a nucleic acid encoding an IL-1 protein.

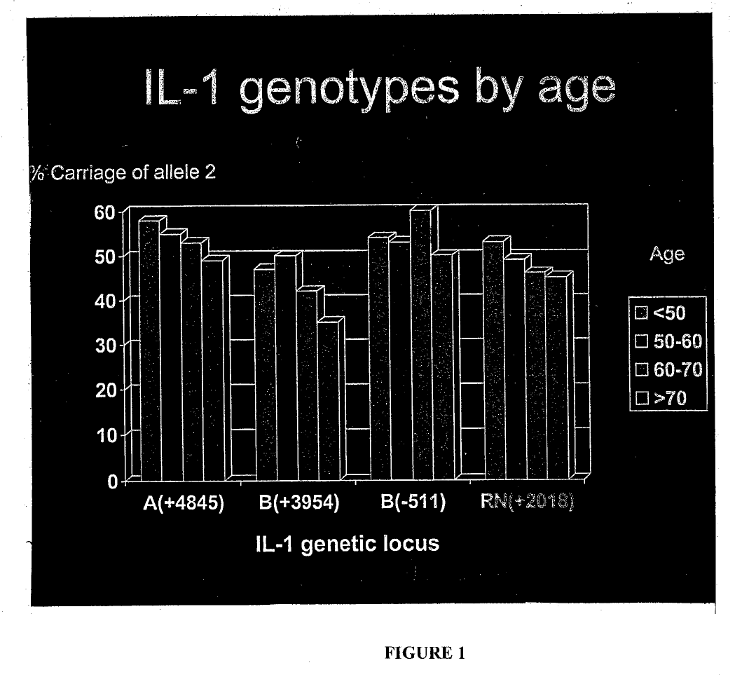

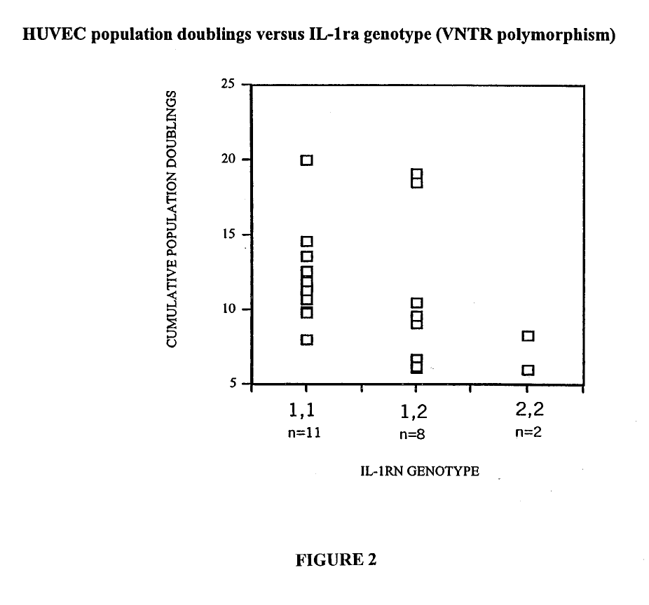

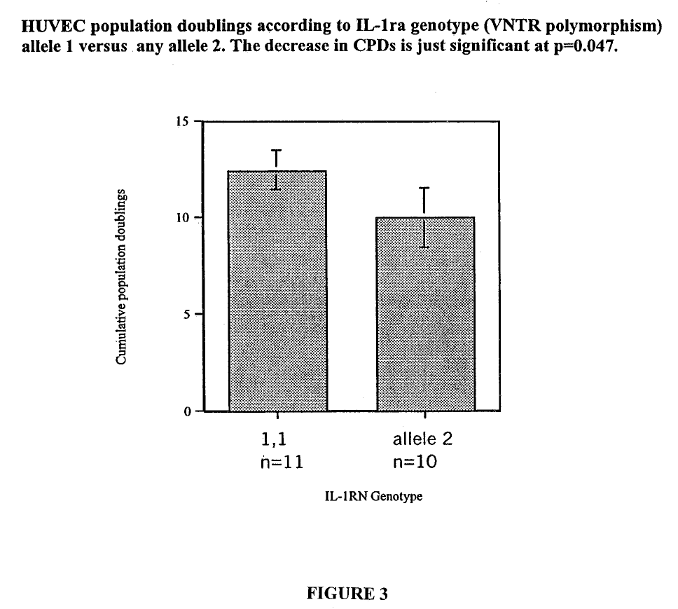

An “IL-1 polypeptide” and “IL-1 protein” are intended to encompass polypeptides comprising the amino acid sequence encoded by the IL-1 genomic DNA sequences shown in FIGS. 1, 2, and 3, or fragments thereof, and homologs thereof and include agonist and antagonist polypeptides.

The “immune system” is a complex system of cells and factors that functions to prevent infection by viruses, bacteria, parasites, helminths, fungi, insects, protozoans etc, and to protect against foreign bodies or non-self material generally. The immune system also functions to destroy damaged or diseased cells of the body, including cancer cells. The immune system further functions to discriminate between self and non-self, and mediates inflammation and systemic shock. Impaired immune system function refers to defects in any of these activities.

The term “including” is used herein to mean “including but not limited to”. “Including” and “including but not limited to” are used interchangeably.

“Increased risk” or “increased susceptibility” refers to a statistically higher frequency of occurrence of the disease or condition in an individual carrying a particular polymorphic allele in comparison to the frequency of occurrence of the disease or condition in a member of a population that does not carry the particular polymorphic allele.

The term “interact” as used herein is meant to include detectable relationships or associations (e.g. biochemical interactions) between molecules, such as interactions between protein-protein, protein-nucleic acid, nucleic acid-nucleic acid and protein-small molecule or nucleic acid-small molecule in nature.

The term “isolated” as used herein with respect to nucleic acids, such as DNA or RNA, refers to molecules separated from other DNAs, or RNAs, respectively, that are present in the natural source of the macromolecule. For example, an isolated nucleic acid encoding one of the subject IL-1 polypeptides preferably includes no more than 10 kilobases (kb) of nucleic acid sequence which naturally immediately flanks the IL-1 gene in genomic DNA, more preferably no more than 5 kb of such naturally occurring flanking sequences, and most preferably less than 1.5 kb of such naturally occurring flanking sequence. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides.

A “knock-in” transgenic animal refers to an animal that has had a modified gene introduced into its genome and the modified gene can be of exogenous or endogenous origin.

A “knock-out” transgenic animal refers to an animal in which there is partial or complete suppression of the expression of an endogenous gene (e.g, based on deletion of at least a portion of the gene, replacement of at least a portion of the gene with a second sequence, introduction of stop codons, the mutation of bases encoding critical amino acids, or the removal of an intron junction, etc.).

A “knock-out construct” refers to a nucleic acid sequence that can be used to decrease or suppress expression of a protein encoded by endogenous DNA sequences in a cell. In a simple example, the knock-out construct is comprised of a gene, such as the IL-1RN gene, with a deletion in a critical portion of the gene so that active protein cannot be expressed therefrom. Alternatively, a number of termination codons can be added to the native gene to cause early termination of the protein or an intron junction can be inactivated. In a typical knock-out construct, some portion of the gene is replaced with a selectable marker (such as the neo gene) so that the gene can be represented as follows: IL-1RN 5′/neo/IL-1RN 3′, where IL-1RN5′ and IL-1 RN 3′, refer to genomic or cDNA sequences which are, respectively, upstream and downstream relative to a portion of the IL-1RN gene and where neo refers to a nEOAycin resistance gene. In another knock-out construct, a second selectable marker is added in a flanking position so that the gene can be represented as: IL-1RN/neo/IL-1RN/TK, where TK is a thymidine kinase gene which can be added to either the IL-1 RN5′ or the IL-1 RN3′ sequence of the preceding construct and which further can be selected against (i.e. is a negative selectable marker) in appropriate media. This two-marker construct allows the selection of homologous recombination events, which removes the flanking TK marker, from non-homologous recombination events which typically retain the TK sequences. The gene deletion and/or replacement can be from the exons, introns, especially intron junctions, and/or the regulatory regions such as promoters.

“Linkage disequilibrium” refers to co-inheritance of two alleles at frequencies greater than would be expected from the separate frequencies of occurrence of each allele in a given control population. The expected frequency of occurrence of two alleles that are inherited independently is the frequency of the first allele multiplied by the frequency of the second allele. Alleles that co-occur at expected frequencies are said to be in “linkage equilibrium”. The cause of linkage disequilibrium is often unclear. It can be due to selection for certain allele combinations or to recent admixture of genetically heterogeneous populations. In addition, in the case of markers that are very tightly linked to a disease gene, an association of an allele (or group of linked alleles) with the disease gene is expected if the disease mutation occurred in the recent past, so that sufficient time has not elapsed for equilibrium to be achieved through recombination events in the specific chromosomal region. When referring to allelic patterns that are comprised of more than one allele, a first allelic pattern is in linkage disequilibrium with a second allelic pattern if all the alleles that comprise the first allelic pattern are in linkage disequilibrium with at least one of the alleles of the second allelic pattern. An example of linkage disequilibrium is that which occurs between the alleles at the IL-1RN (+2018) and IL-1RN (VNTR) polymorphic sites. The two alleles at IL-1RN (+2018) are 100% in linkage disequilibrium with the two most frequent alleles of IL-1RN (VNTR), which are allele 1 and allele 2.

The term “marker” refers to a sequence in the genome that is known to vary among individuals. For example, the IL-1RN gene has a marker that consists of a variable number of tandem repeats (VNTR). The different sequence variants at a given marker are called alleles, mutations or polymorphisms. For example, the VNTR marker has at least five different alleles, three of which are rare. Different alleles could have a single base change, including substitution, insertion or deletion, or could have a change that affects multiple bases, including substitutions, insertions, deletions, repeats, inversions and combinations thereof.

“Modulate” refers to the ability of a substance to regulate bioactivity. When applied to an IL-1 bioactivity, an agonist or antagonist can modulate bioactivity for example by agonizing or antagonizing an IL-1 synthesis, receptor interaction, or IL-1 mediated signal transduction mechanism.

A “mutated gene” or “mutation” or “functional mutation” refers to an allelic form of a gene, which is capable of altering the phenotype of a subject having the mutated gene relative to a subject which does not have the mutated gene. The altered phenotype caused by a mutation can be corrected or compensated for by certain agents. If a subject must be homozygous for this mutation to have an altered phenotype, the mutation is said to be recessive. If one copy of the mutated gene is sufficient to alter the phenotype of the subject, the mutation is said to be dominant. If a subject has one copy of the mutated gene and has a phenotype that is intermediate between that of a homozygous and that of a heterozygous subject (for that gene), the mutation is said to be co-dominant.

“Neurological function” refers to all the activities of the nervous system, including the central nervous system, the peripheral nervous system, the autonomic nervous system and the production of hormones and factors by neural cells and cells regulated by neural cells.

A “non-human animal” of the invention includes mammals such as rodents, non-human primates, sheep, dogs, cows, goats, etc. Preferred non-human animals are selected from the rodent family including rat and mouse, most preferably mouse, though transgenic amphibians, such as members of the Xenopus genus, and transgenic chickens can also provide important tools for understanding and identifying agents which can affect, for example, embryogenesis and tissue formation. The term “chimeric animal” is used herein to refer to animals in which the recombinant gene is found, or in which the recombinant gene is expressed in some but not all cells of the animal. The term “tissue-specific chimeric animal” indicates that one of the recombinant IL-1 genes is present and/or expressed or disrupted in some tissues but not others. The term “non-human mammal” refers to any members of the class Mammalia, except for humans.

As used herein, the term “nucleic acid” refers to polynucleotides or oligonucleotides such as deoxyribonucleic acid (DNA), and, where appropriate, ribonucleic acid (RNA). The term should also be understood to include, as equivalents, analogs of either RNA or DNA made from nucleotide analogs (e.g. peptide nucleic acids) and as applicable to the embodiment being described, single (sense or antisense) and double-stranded polynucleotides.

“Nutraceuticals” are defined as substances comprising vitamins, minerals, proteins, amino acids, sugars, phytoestrogens, flavonoids, phenolics, anthocyanins, carotenoids, polymers of the above, and mixtures of the above.

The term “or” as used herein should be understood to mean “and/or”, unless the context clearly indicates otherwise.

The term “polymorphism” refers to the coexistence of more than one form of a gene or portion (e.g., allelic variant) thereof. A portion of a gene of which there are at least two different forms, i.e., two different nucleotide sequences, is referred to as a “polymorphic region.” As used herein, the term “polymorphic region” includes, without limitation, a polymorphic site consisting of a single nucleotide, e.g., a single nucleotide polymorphism (SNP). A specific genetic sequence at a polymorphic region is an allele. A polymorphic region can be a single nucleotide, the identity of which differs in different alleles. A polymorphic region can also be more than one nucleotide long, and possibly significantly longer in length.

The term “propensity” as used herein in reference to a condition or disease state, as in “propensity” for a condition or disease, is used interchangeably with the expressions “susceptibility” or “predisposition”. The term “propensity” as used in reference to a condition or disease state indicates that an individual is at increased risk for the future development of a condition or disease. For example, if an allele is discovered to be associated with or predictive of a particular disease or condition, an individual carrying the allele has a greater propensity for developing the particular disease or condition.

“Reactive oxygen species” include peroxides, oxygen radicals, carbon radicals resulting from oxidation, superoxides and metal radicals resulting from oxidation.

“Small molecule” as used herein, is meant to refer to a composition, which has a molecular weight of less than about 5 kD and most preferably less than about 4 kD. Small molecules can be nucleic acids, peptides, peptidomimetics, carbohydrates, lipids or other organic or inorganic molecules.

As used herein, the term “specifically hybridizes” or “specifically detects” refers to the ability of a nucleic acid molecule to hybridize to at least approximately 6 consecutive nucleotides of a sample nucleic acid.

“Transcriptional regulatory sequence” is a generic term used throughout the specification to refer to DNA sequences, such as initiation signals, enhancers, and promoters, which induce or control transcription of protein coding sequences with which they are operably linked.

As used herein, the term “transgene” means a nucleic acid sequence (encoding, e.g., one of the IL-1 polypeptides, or an antisense transcript thereto) which has been introduced into a cell. A transgene could be partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). A transgene can also be present in a cell in the form of an episome. A transgene can include one or more transcriptional regulatory sequences and any other nucleic acid, such as introns, that may be necessary for optimal expression of a selected nucleic acid.

A “transgenic animal” refers to any animal, preferably a non-human mammal, bird or an amphibian, in which one or more of the cells of the animal contain heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. In the typical transgenic animals described herein, the transgene causes cells to express a recombinant form of one of an IL-1 polypeptide, e.g. either agonistic or antagonistic forms. However, transgenic animals in which the recombinant gene is silent are also contemplated, as for example, the FLP or CRE recombinase dependent constructs described below. Moreover, “transgenic animal” also includes those recombinant animals in which gene disruption of one or more genes is caused by human intervention, including both recombination and antisense techniques. The term is intended to include all progeny generations. Thus, the founder animal and all F1, F2, F3, and so on, progeny thereof are included.

The term “treating” as used herein is intended to encompass curing as well as ameliorating at least one symptom of a disease or at least one abnormality associated with a disorder.

The term “vector” refers to a nucleic acid molecule, which is capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

The term “wild-type allele” refers to an allele of a gene which, when present in two copies in a subject results in a wild-type phenotype. There can be several different wild-type alleles of a specific gene, since certain nucleotide changes in a gene may not affect the phenotype of a subject having two copies of the gene with the nucleotide changes.

Predictive Medicine

Polymorphisms Associated with Senescence

The present invention is based at least in part, on the identification of alleles that are associated with the earlier onset of aging-related conditions. Therefore, detection of these alleles, alone or in conjunction with another means in a subject indicate that the subject has or is predisposed to EOA. For example, IL-1 polymorphic alleles which are associated with EOA include allele 2 of each of the following markers: IL-1A (+4845), IL-1B (+3954) and IL-1RN (VNTR) or an allele that is in linkage disequilibrium with one of the aforementioned alleles. In particularly preferred embodiments, the presence of a particular allelic pattern of one or more of the above mentioned IL-1 polymorphic loci is used to predict the susceptibility of an individual to developing EOA. In particular, there are three patterns of alleles at loci in the IL-1 gene cluster that show various associations with EOA. These patterns are referred to herein as patterns 1, 2 and 3. In a preferred embodiment, this detection of any of these patterns provides information about the likelihood that the subject will have EOA. Pattern 1 is associated with a shortened lifespan. Detection of pattern 2 indicates that a subject has a predisposition to earlier cellular senescence.

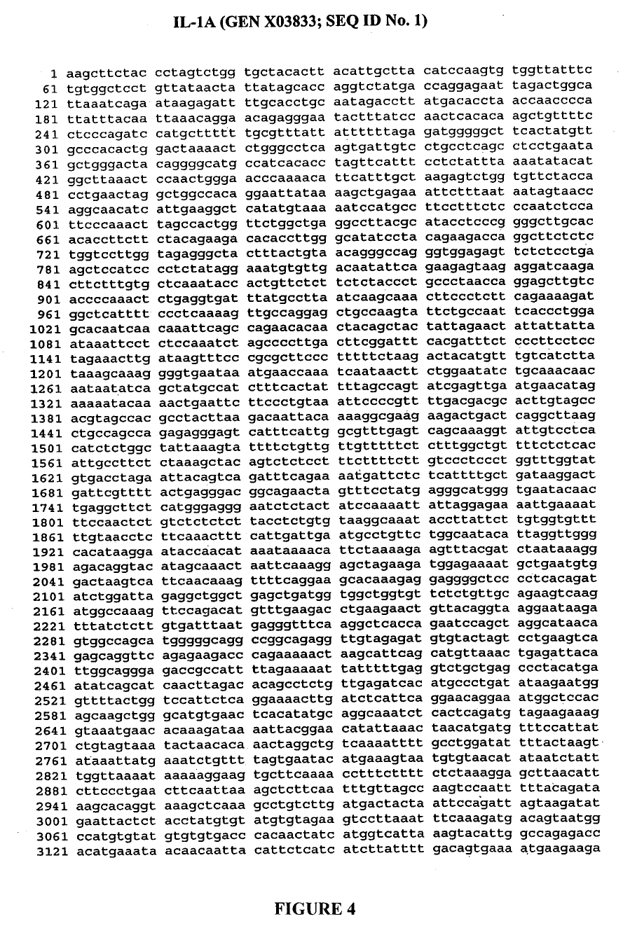

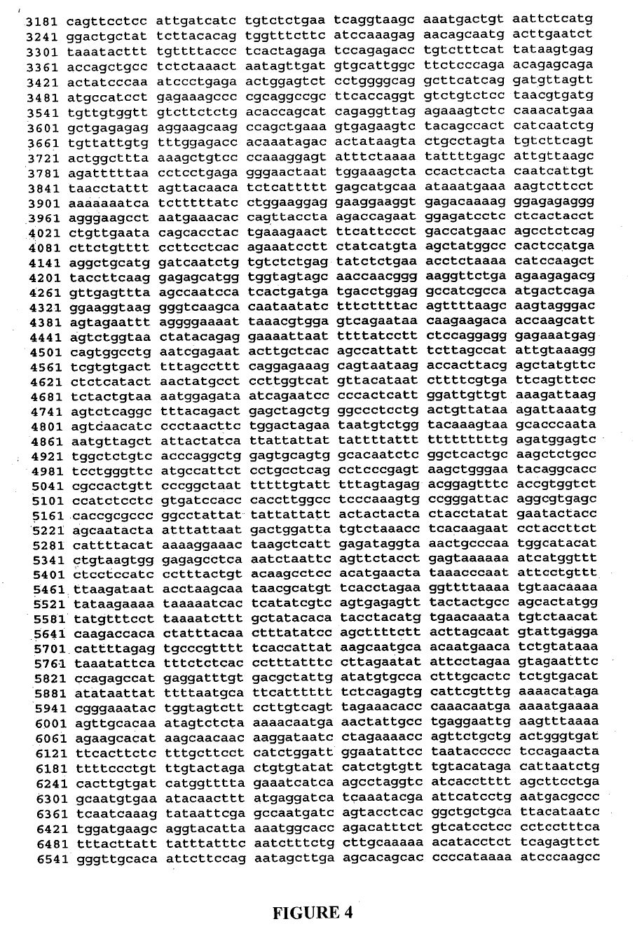

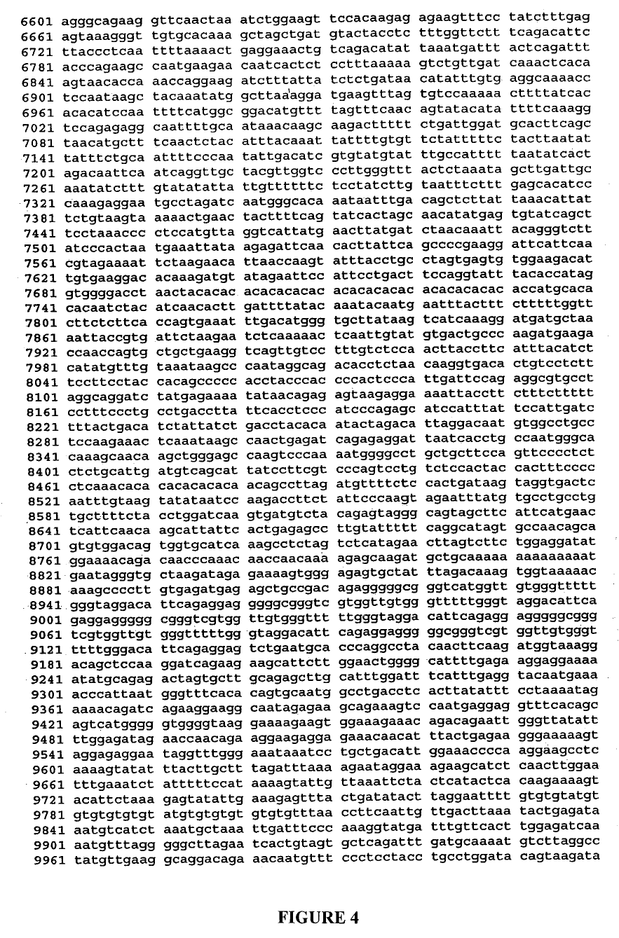

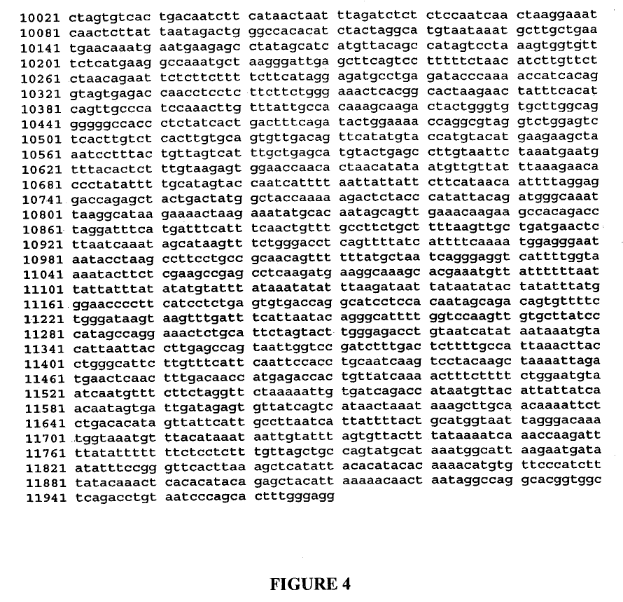

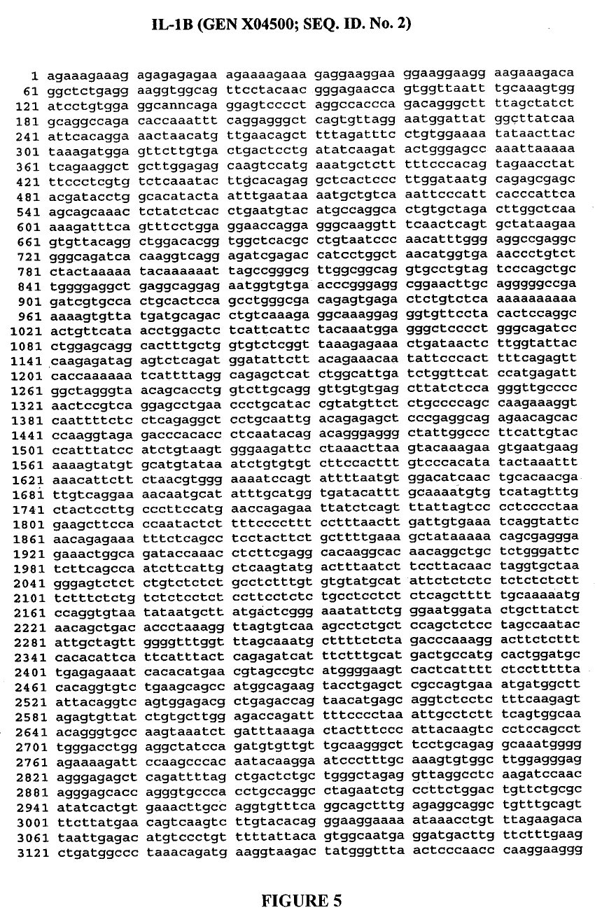

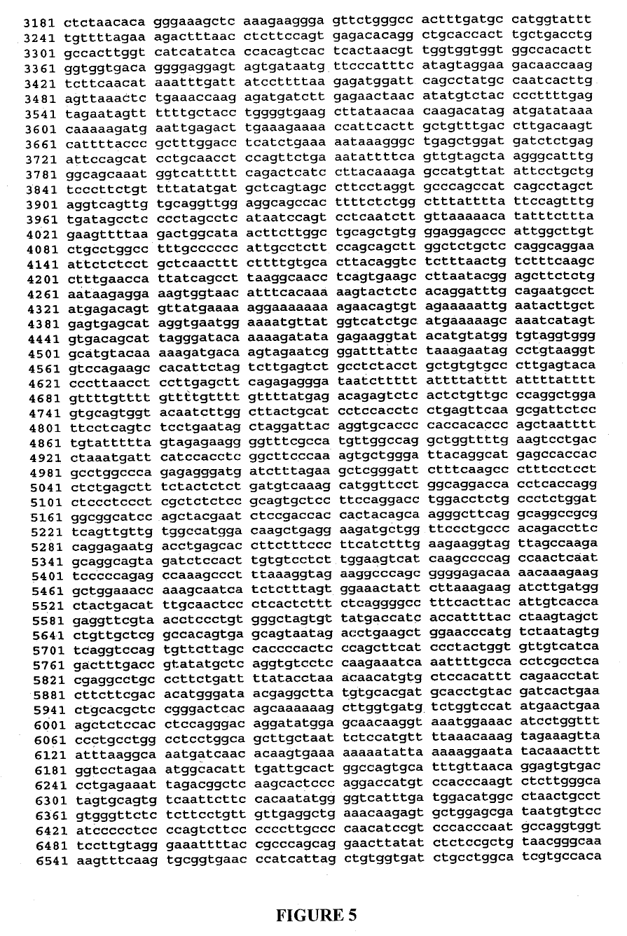

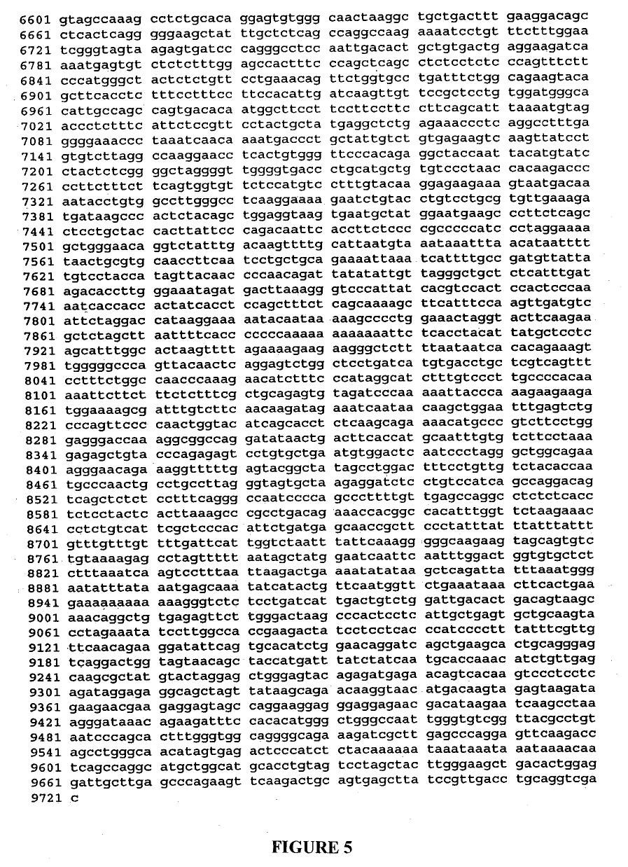

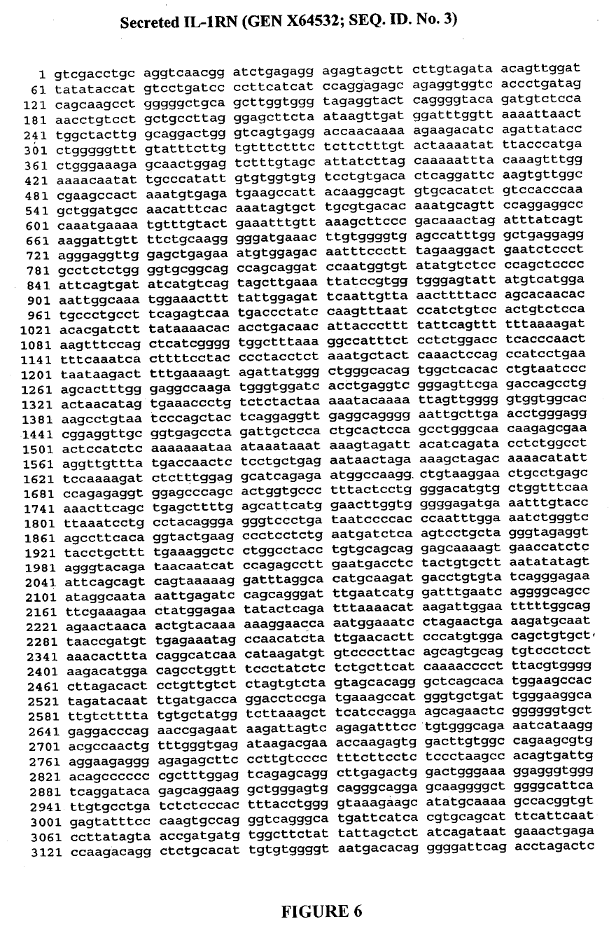

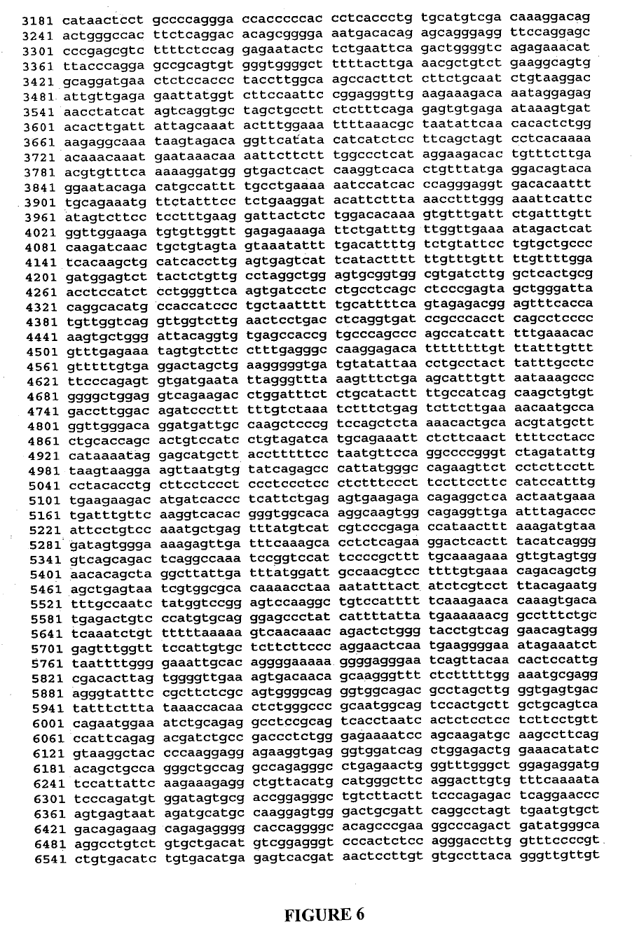

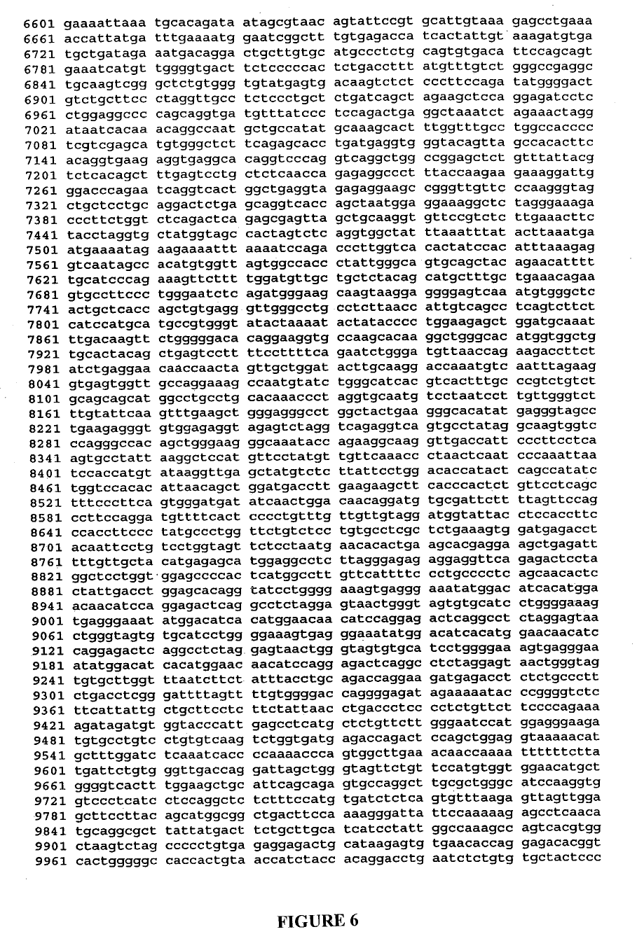

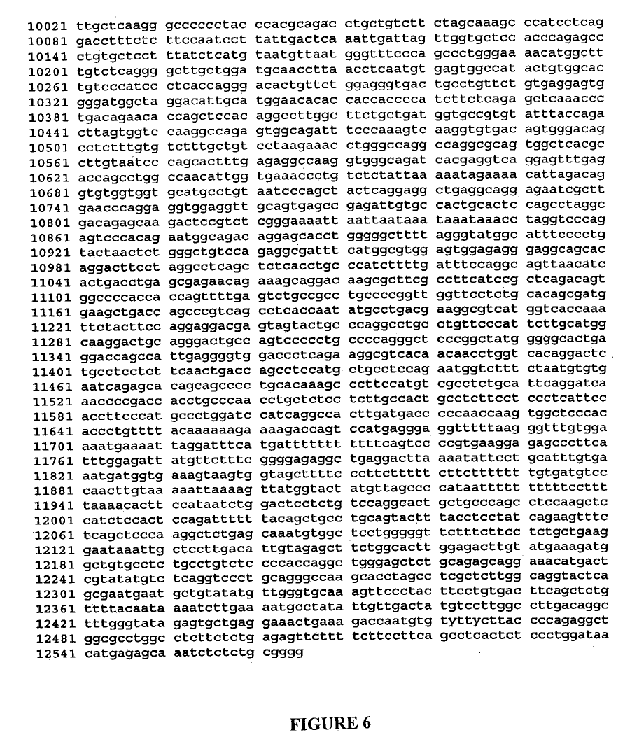

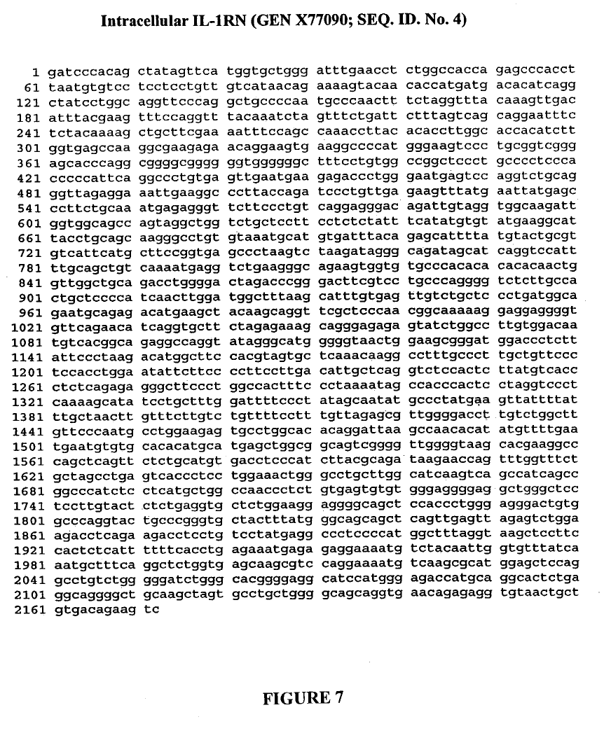

These IL-1 locus polymorphisms represent single base variations within the IL-1A/IL-1B/IL-1RN gene cluster (see FIG. 4). The IL-1A (+4845) polymorphism is a single base variation (allele 1 is G, allele 2 is T) at position +4845 within Exon V of the IL-1A gene which encodes the inflammatory cytokine IL-1a (Gubler, et al.(1989) Interleukin, inflammation and disease (Bbmford and Henderson, eds.) p.31-45, Elsevier publishers; and Van den velden and Reitsma (1993) Hum Mol Genetics 2:1753-50). The IL-1A (+4845) polymorphism occurs in the coding region of the gene and results in a single amino acid variation in the encoded protein (Van den Velden and Reitsma (1993) Hum Mol Genet 2: 1753). The IL-1B (+3954) polymorphism was first described as a Taq I restriction fragment length polymorphism (RFLP) (Pociot et al. (1992) Eur J Clin Invest 22: 396-402) and has subsequently been characterized as a single base variation (allele 1 is C, allele 2 is T) at position +3954 in Exon V of the IL-1B gene (di Giovine et al. (1995) Cytokine 7: 600-606). This single nucleotide change in the open reading frame of IL-1B does not appear to qualitatively affect the sequence of the encoded IL-1 beta polypeptide because it occurs at the third position of a TTC phenylalanine codon (F) of allele 1 and therefore allele 2 merely substitutes a TTT phenylalanine codon at this position which encodes amino acid 105 of the IL-1B gene product. In addition, the IL-1RN (+2018) polymorphism (Clay et al. (1996) Hum Genet 97: 723-26) is a single base variation (allele 1 is T, allele 2 is C), also referred to as exon 2 (8006) (GenBank:X64532 at 8006). Finally, the IL-RN variable number of tandem repeats (VNTR) polymorphism occurs within the second intron the IL-1 receptor antagonist encoding gene (Steinkasserer (1991) Nucleic Acids Res 19: 5090-5). Allele 2 of the of the IL-1RN (VNTR) polymorphism corresponds to two repeats of an 86-base pair sequence, while allele 1 corresponds to four repeats, allele 3 to three repeats, allele 4 to five repeats, and allele 5 to six repeats (Tarlow et al. (1993) Hum Genet 91: 403-4). Detection of any one of these IL-1 allelic variants in an individual suggests that a subject has increased susceptibility to the early onset of aging-related conditions.

However, because these alleles are in linkage disequilibrium with other alleles, the detection of such other linked alleles can also indicate that that a subject has increased susceptibility to the early onset of aging-related conditions. For example, the following alleles of the IL-1 (33221461) haplotype are in linkage disequilibrium:

allele 3 of the 222/223 marker of IL-1A
allele 3 of the gz5/gz6 marker of IL-1A
allele 2 of the −889 marker of IL-1A
allele 2 of the +3954 marker of IL-1B
allele 1 of the −3737 marker of IL-1B
allele 1 of the −511 marker of IL-1B
allele 4 of the gaat.p33330 marker
allele 6 of the Y31 marker
allele 1 of the VNTR or (+2018) marker of IL-1RN

Therefore, allele 1 of IL-1B (−511) and allele 1 of IL-1 RN (VNTR) are in strong linkage disequilibrium with one another and each of these is in linkage disequilibrium with allele 1 of the −511 marker of IL-1B. Furthermore, in alternative embodiments of the present invention, genotyping analysis at the 222/223 marker of IL-1A, the gz5/gz6 marker of IL-1A, the −889 marker of IL-1A, the +3954 marker of IL-1B, the gaat.p33330 marker of the IL-1B/IL-1RN intergenic region, or the Y31 marker of the IL-1B/IL-1RN intergenic region is determined, and the presence of a polymorphic allele which is linked to one or more of the preferred EOA-predictive alleles is detected.

In addition, allele 2 of the IL-1RN (+2018) polymorphism is known to be in linkage disequilibrium with allele 2 of the IL-1RN (VNTR) polymorphic locus, which in turn is a part of the 44112332 human haplotype. The 44112332 haplotype comprises the following genotype:

allele 4 of the 222/223 marker of IL-1A
allele 4 of the gz5/gz6 marker of IL-1A
allele 1 of the −889 marker of IL-1A
allele 1 of the +3954 marker of IL-1B
allele 1 of the −3737 marker of IL-1B
allele 2 of the −511 marker of IL-1B
allele 3 of the gaat.p33330 marker
allele 3 of the Y31 marker
allele 2 of the VNTR marker of IL-1RN

Similarly, three other polymorphisms in an IL-1RN alternative exon (Exon 1ic, which produces an intracellular form of the gene product) are also in linkage disequilibrium with allele 2 of IL-1RN (VNTR) (Clay et al. (1996) Hum Genet 97: 723-26). These include: the IL-1RN exon 1ic (1812) polymorphism (GenBank:X77090 at 1812); the IL-1RN exon 1ic (1868) polymorphism (GenBank:X77090 at 1868); and the IL-1RN exon 1ic (1887) polymorphism (GenBank:X77090 at 1887). Furthermore yet another polymorphism in the promoter for the alternatively spliced intracellular form of the gene, the Pic (1731) polymorphism (GenBank:X77090 at 1731), is also in linkage disequilibrium with allele 2 of the IL-1RN (VNTR) polymorphic locus (Clay et al. (1996) Hum Genet 97: 723-26). The corresponding sequence alterations for each of these IL-1RN polymorphic loci is shown below.

| | Exon 2 | Exon 1ic −1 | Exon 1ic −2 | Exon 1ic −3 | Pic |
|---|---|---|---|---|---|
| | (+2018 of | (1812 of | (1868 of | (1887 of | (1731 of |
| Allele # | IL-1RN) | GB: X77090) | GB: X77090 | GB: X77090) | GB: X77090) |
| 1 | T | G | A | G | G |
| 2 | C | A | G | C | A |

For each of these polymorphic loci, the allele 1 sequence variant has been determined to be in linkage disequilibrium with allele 1 of the IL-1RN (VNTR) locus (Clay et al. (1996) Hum Genet 97: 723-26).

A third haplotype, termed pattern 3, comprises the following alleles in linkage disequilibrium:

allele 1 of the −889 marker of IL-1A
allele 1 of the +4845 marker of IL-1A
allele 1 of the +3954 marker of IL-1B
allele 1 of the −511 marker of IL-1B
allele 2 of the −3737 marker of IL-1B
allele 1 of the +2018 marker of IL-1RN
allele 1 of the VNTR marker of IL-1RN

Pattern 3 appears to give the lowest inflammation of the 3 patterns. In one study, 239 subjects<age 60 were evaluated for C-reactive protein (CRP) levels. Individuals with unambiguous Pattern 1 had CRP 75% higher than Pattern 3. Individuals with unambiguous Pattern 2 had CRP 11-26% higher than Pattern 3. Subjects with Pattern 3 are at increased risk for coronary artery restenosis following angioplasty and stents. In addition, it is expected that individuals with Pattern 3 may exhibit the following:

i. Reduced risk for early chronic diseases.

ii. Adverse reactions to anti-inflammatory agents.

iii. Increased risk for infectious diseases and their complications.

In addition to the allelic patterns described above, one of skill in the art can, in view of this specification, readily identify other alleles (including polymorphisms and mutations) that are in linkage disequilibrium with an allele associated with EOA. For example, a nucleic acid sample from a first group of subjects without known EOA associated alleles can be collected, as well as DNA from a second group of subjects carrying one or more EOA associated alleles. The nucleic acid sample can then be compared to identify those alleles that are over-represented in the second group as compared with the first group, wherein such alleles are presumably associated with EOA. Alternatively, alleles that are in linkage disequilibrium with a EOA associated allele can be identified, for example, by genotyping a large population and performing statistical analysis to determine which alleles appear more commonly together than expected. Preferably the group is chosen to be comprised of genetically related individuals. Genetically related individuals include individuals from the same race, the same ethnic group, or even the same family. As the degree of genetic relatedness between a control group and a test group increases, so does the predictive value of polymorphic alleles which are ever more distantly linked to a disease-causing allele. This is because less evolutionary time has passed to allow polymorphisms which are linked along a chromosome in a founder population to redistribute through genetic cross-over events. Thus race-specific, ethnic-specific, and even family-specific diagnostic genotyping assays can be developed to allow for the detection of disease alleles which arose at ever more recent times in human evolution, e.g., after divergence of the major human races, after the separation of human populations into distinct ethnic groups, and even within the recent history of a particular family line.

Linkage disequilibrium between two polymorphic markers or between one polymorphic marker and a disease-causing mutation is a meta-stable state. Absent selective pressure or the sporadic linked reoccurrence of the underlying mutational events, the polymorphisms will =eventually become disassociated by chromosomal recombination events and will thereby reach linkage equilibrium through the course of human evolution. Thus, the likelihood of finding a polymorphic allele in linkage disequilibrium with a disease or condition may increase with changes in at least two factors: decreasing physical distance between the polymorphic marker and the disease-causing mutation, and decreasing number of meiotic generations available for the dissociation of the linked pair. Consideration of the latter factor suggests that, the more closely related two individuals are, the more likely they will share a common parental chromosome or chromosomal region containing the linked polymorphisms and the less likely that this linked pair will have become unlinked through meiotic cross-over events occurring each generation. As a result, the more closely related two individuals are, the more likely it is that widely spaced polymorphisms may be co-inherited. Thus, for individuals related by common race, ethnicity or family, the reliability of ever more distantly spaced polymorphic loci can be relied upon as an indicator of inheritance of a linked disease-causing mutation.

Appropriate probes may be designed to hybridize to a specific gene of the IL-1 locus, such as IL-1A, IL-1B or IL-1RN or a related gene. These genomic DNA sequences are shown in FIGS. 1, 2 and 3, respectively, and further correspond to formal SEQ ID Nos. 15, 16 and 17, respectively. Alternatively, these probes may incorporate at other regions of the relevant genomic locus, including intergenic sequences. Indeed the IL-1 region of human chromosome 2 spans some 400,000 base pairs and, assuming an average of one single nucleotide polymorphism every 1,000 base pairs, includes some 400 SNPs loci alone. Yet other polymorphisms available for use with the immediate invention are obtainable from various public sources. For example, the human genome database collects intragenic SNPs, is searchable by sequence and currently contains approximately 2,700 entries (http://hgbase.interactiva.de). Also available is a human polymorphism database maintained by the Massachusetts Institute of Technology (MIT SNP database (http://www.genome.wi.mit.edu/SNP/human/index.html)). From such sources SNPs as well as other human polymorphisms may be found.

For example, examination of the IL-1 region of the human genome in any one of these databases reveals that the IL-1 locus genes are flanked by a centromere proximal polymorphic marker designated microsatellite marker AFM220ze3 at 127.4 cM (centiMorgans) (see GenBank Acc. No. Z17008) and a distal polymorphic marker designated microsatellite anchor marker AFMO87xal at 127.9 cM (see GenBank Acc. No. Z16545). These human polymorphic loci are both CA dinucleotide repeat microsatellite polymorphisms, and, as such, show a high degree of heterozygosity in human populations. For example, one allele of AFM220ze3 generates a 211 bp PCR amplification product with a 5′ primer of the sequence TGTACCTAAGCCCACCCTTTAGAGC (SEQ ID No. 18) and a 3′ primer of the sequence TGGCCTCCAGAAACCTCCAA (SEQ ID No. 19). Furthermore, one allele of AFM087xa1 generates a 177 bp PCR amplification product with a 5′ primer of the sequence GCTGATATTCTGGTGGGAAA (SEQ ID No.20) and a 3′ primer of the sequence GGCAAGAGCAAAACTCTGTC (SEQ ID No. 21). Equivalent primers corresponding to unique sequences occurring 5′ and 3′ to these human chromosome 2 CA dinucleotide repeat polymorphisms will be apparent to one of skill in the art. Reasonable equivalent primers include those which hybridize within about 1 kb of the designated primer, and which further are anywhere from about 17 bp to about 27 bp in length. A general guideline for designing primers for amplification of unique human chromosomal genomic sequences is that they possess a melting temperature of at least about 50 C, wherein an approximate melting temperature can be estimated using the formula Tₘₑₗₜ=[2× (# of A or T)+4×(# of G or C)].

A number of other human polymorphic loci occur between these two CA dinucleotide repeat polymorphisms and provide additional targets for determination of a EOA prognostic allele in a family or other group of genetically related individuals. For example, the National Center for Biotechnology Information web site (www.ncbi.nlm.nih.gov/genemap/) lists a number of polymorphism markers in the region of the IL-1 locus and provides guidance in designing appropriate primers for amplification and analysis of these markers.

Accordingly, the nucleotide segments of the invention may be used for their ability to selectively form duplex molecules with complementary stretches of human chromosome 2 q 12-13 or cDNAs from that region or to provide primers for amplification of DNA or cDNA from this region. The design of appropriate probes for this purpose requires consideration of a number of factors. For example, fragments having a length of between 10, 15, or 18 nucleotides to about 20, or to about 30 nucleotides, will find particular utility. Longer sequences, e.g., 40, 50, 80, 90, 100, even up to full length, are even more preferred for certain embodiments. Lengths of oligonucleotides of at least about 18 to 20 nucleotides are well accepted by those of skill in the art as sufficient to allow sufficiently specific hybridization so as to be useful as a molecular probe. Furthermore, depending on the application envisioned, one will desire to employ varying conditions of hybridization to achieve varying degrees of selectivity of probe towards target sequence. For applications requiring high selectivity, one will typically desire to employ relatively stringent conditions to form the hybrids. For example, relatively low salt and/or high temperature conditions, such as provided by 0.02 M-0.15M NaCl at temperatures of about 50 C to about 70 C. Such selective conditions may tolerate little, if any, mismatch between the probe and the template or target strand.

Other alleles or other indicia of EOA can be detected or monitored in a subject in conjunction with detection of the alleles described above.

Detection of Alleles

Many methods are available for detecting specific alleles at human polymorphic loci. The preferred method for detecting a specific polymorphic allele will depend, in part, upon the molecular nature of the polymorphism. For example, the various allelic forms of the polymorphic locus may differ by a single base-pair of the DNA. Such single nucleotide polymorphisms (or SNPs) are major contributors to genetic variation, comprising some 80% of all known polymorphisms, and their density in the human genome is estimated to be on average 1 per 1,000 base pairs. SNPs are most frequently biallelic-occurring in only two different forms (although up to four different forms of an SNP, corresponding to the four different nucleotide bases occurring in DNA, are theoretically possible). Nevertheless, SNPs are mutationally more stable than other polymorphisms, making them suitable for association studies in which linkage disequilibrium between markers and an unknown variant is used to map disease-causing mutations. In addition, because SNPs typically have only two alleles, they can be genotyped by a simple plus/minus assay rather than a length measurement, making them more amenable to automation.

A variety of methods are available for detecting the presence of a particular single nucleotide polymorphic allele in an individual. Advancements in this field have provided accurate, easy, and inexpensive large-scale SNP genotyping. Most recently, for example, several new techniques have been described including dynamic allele-specific hybridization (DASH), microplate array diagonal gel electrophoresis (MADGE), pyrosequencing, oligonucleotide-specific ligation, the TaqMan system as well as various DNA “chip” technologies such as the Affymetrix SNP chips. These methods require amplification of the target genetic region, typically by PCR. Still other newly developed methods, based on the generation of small signal molecules by invasive cleavage followed by mass spectrometry or immobilized padlock probes and rolling-circle amplification, might eventually eliminate the need for PCR. Several of the methods known in the art for detecting specific single nucleotide polymorphisms are summarized below. The method of the present invention is understood to include all available methods.

Several methods have been developed to facilitate analysis of single nucleotide polymorphisms. In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of a polymorphic site. Cohen, D. et al. (French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA™ is described by Goelet, P. et al. (PCT Appln. No. 92/15712). The method of Goelet, P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet, P. et al. is preferably a heterogeneous phase assay, in which the primer or the target molecule is immobilized to a solid phase.

Recently, several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvanen, A. -C., et al., Genomics 8:684-692 (1990); Kuppuswamy, M. N. et al., Proc. Natl. Acad. Sci. (U.S.A.) 88:1143-1147 (1991); Prezant, T. R. et al., Hum. Mutat. 1:159-164 (1992); Ugozzoli, L. et al., GATA 9:107-112 (1992); Nyren, P. et al., Anal. Biochem. 208:171-175 (1993)). These methods differ from GBA™ in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A. -C., et al., Amer. J. Hum. Genet. 52:46-59 (1993)).

For mutations that produce premature termination of protein translation, the protein truncation test (PTT) offers an efficient diagnostic approach (Roest, et. al., (1993) Hum. Mol Genet. 2:1719-21; van der Luijt, et. al., (1994) Genomics 20:1-4). For PTT, RNA is initially isolated from available tissue and reverse-transcribed, and the segment of interest is amplified by PCR. The products of reverse transcription PCR are then used as a template for nested PCR amplification with a primer that contains an RNA polymerase promoter and a sequence for initiating eukaryotic translation. After amplification of the region of interest, the unique motifs incorporated into the primer permit sequential in vitro transcription and translation of the PCR products. Upon sodium dodecyl sulfate-polyacrylamide gel electrophoresis of translation products, the appearance of truncated polypeptides signals the presence of a mutation that causes premature termination of translation. In a variation of this technique, DNA (as opposed to RNA) is used as a PCR template when the target region of interest is derived from a single exon.

Any cell type or tissue may be utilized to obtain nucleic acid samples for use in the diagnostics described herein. In a preferred embodiment, the DNA sample is obtained from a bodily fluid, e.g, blood, obtained by known techniques (e.g. venipuncture) or saliva. Alternatively, nucleic acid tests can be performed on dry samples (e.g. hair or skin). When using RNA or protein, the cells or tissues that may be utilized must express an IL-1 gene.

Diagnostic procedures may also be performed in situ directly upon tissue sections (fixed and/or frozen) of patient tissue obtained from biopsies or resections, such that no nucleic acid purification is necessary. Nucleic acid reagents may be used as probes and/or primers for such in situ procedures (see, for example, Nuovo, G. J., 1992, PCR in situ hybridization: protocols and applications, Raven Press, NY).

In addition to methods which focus primarily on the detection of one nucleic acid sequence, profiles may also be assessed in such detection schemes. Fingerprint profiles may be generated, for example, by utilizing a differential display procedure, Northern analysis and/or RT-PCR.

A preferred detection method is allele specific hybridization using probes overlapping a region of at least one allele of an IL-1 proinflammatory haplotype and having about 5, 10, 20, 25, or 30 nucleotides around the mutation or polymorphic region. In a preferred embodiment of the invention, several probes capable of hybridizing specifically to other allelic variants involved in a EOA are attached to a solid phase support, e.g., a “chip” (which can hold up to about 250,000 oligonucleotides). Oligonucleotides can be bound to a solid support by a variety of processes, including lithography. Mutation detection analysis using these chips comprising oligonucleotides, also termed “DNA probe arrays” is described e.g., in Cronin et al. (1996) Human Mutation 7:244. In one embodiment, a chip comprises all the allelic variants of at least one polymorphic region of a gene. The solid phase support is then contacted with a test nucleic acid and hybridization to the specific probes is detected. Accordingly, the identity of numerous allelic variants of one or more genes can be identified in a simple hybridization experiment.

These techniques may also comprise the step of amplifying the nucleic acid before analysis. Amplification techniques are known to those of skill in the art and include, but are not limited to cloning, polymerase chain reaction (PCR), polymerase chain reaction of specific alleles (ASA), ligase chain reaction (LCR), nested polymerase chain reaction, self sustained sequence replication (Guatelli, J. C. et al., 1990, Proc. Natl. Acad. Sci. USA 87:1874-1878), transcriptional amplification system (Kwoh, D. Y. et al., 1989, Proc. Natl. Acad. Sci. USA 86:1173-1177), and Q-Beta Replicase (Lizardi, P. M. et al., 1988, Bio/Technology 6:1197).

Amplification products may be assayed in a variety of ways, including size analysis, restriction digestion followed by size analysis, detecting specific tagged oligonucleotide primers in the reaction products, allele-specific oligonucleotide (ASO) hybridization, allele specific 5′ exonuclease detection, sequencing, hybridization, and the like.

PCR based detection means can include multiplex amplification of a plurality of markers simultaneously. For example, it is well known in the art to select PCR primers to generate PCR products that do not overlap in size and can be analyzed simultaneously. Alternatively, it is possible to amplify different markers with primers that are differentially labeled and thus can each be differentially detected. Of course, hybridization based detection means allow the differential detection of multiple PCR products in a sample. Other techniques are known in the art to allow multiplex analyses of a plurality of markers.

In a merely illustrative embodiment, the method includes the steps of (i) collecting a sample of cells from a patient, (ii) isolating nucleic acid (e.g., genomic, mRNA or both) from the cells of the sample, (iii) contacting the nucleic acid sample with one or more primers which specifically hybridize 5′ and 3′ to at least one allele of an IL-1 proinflammatory haplotype under conditions such that hybridization and amplification of the allele occurs, and (iv) detecting the amplification product. These detection schemes are especially useful for the detection of nucleic acid molecules if such molecules are present in very low numbers.

In a preferred embodiment of the subject assay, the allele of an IL-1 proinflammatory haplotype is identified by alterations in restriction enzyme cleavage patterns. For example, sample and control DNA is isolated, amplified (optionally), digested with one or more restriction endonucleases, and fragment length sizes are determined by gel electrophoresis.

In yet another embodiment, any of a variety of sequencing reactions known in the art can be used to directly sequence the allele. Exemplary sequencing reactions include those based on techniques developed by Maxim and Gilbert ((1977) Proc. Natl Acad Sci USA 74:560) or Sanger (Sanger et al (1977) Proc. Nat. Acad. Sci USA 74:5463). It is also contemplated that any of a variety of automated sequencing procedures may be utilized when performing the subject assays (see, for example Biotechniques (1995) 19:448), including sequencing by mass spectrometry (see, for example PCT publication WO 94/16101; Cohen et al. (1996) Adv Chromatogr 36:127-162; and Griffin et al. (1993) Appl Biochem Biotechnol 38:147-159). It will be evident to one of skill in the art that, for certain embodiments, the occurrence of only one, two or three of the nucleic acid bases need be determined in the sequencing reaction. For instance, A-track or the like, e.g., where only one nucleic acid is detected, can be carried out.

In a further embodiment, protection from cleavage agents (such as a nuclease, hydroxylamine or osmium tetroxide and with piperidine) can be used to detect mismatched bases in RNA/RNA or RNA/DNA or DNA/DNA heteroduplexes (Myers, et al. (1985) Science 230:1242). In general, the art technique of “mismatch cleavage” starts by providing heteroduplexes formed by hybridizing (labeled) RNA or DNA containing the wild-type allele with the sample. The double-stranded duplexes are treated with an agent which cleaves single-stranded regions of the duplex such as which will exist due to base pair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al (1988) Proc. Natl Acad Sci USA 85:4397; and Saleeba et al (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes). For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on an allele of an IL-1 locus haplotype is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify an IL-1 locus allele. For example, single strand conformation polymorphism (SSCP) may be used to detect differences in electrophoretic mobility between mutant and wild type nucleic acids (Orita et al. (1989) Proc Natl. Acad. Sci USA 86:2766, see also Cotton (1993) Mutat Res 285:125-144; and Hayashi (1992) Genet Anal Tech Appl 9:73-79). Single-stranded DNA fragments of sample and control IL-1 locus alleles are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment, the movement of alleles in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:12753).

Examples of other techniques for detecting alleles include, but are not limited to, selective oligonucleotide hybridization, selective amplification, or selective primer extension. For example, oligonucleotide primers may be prepared in which the known mutation or nucleotide difference (e.g., in allelic variants) is placed centrally and then hybridized to target DNA under conditions which permit hybridization only if a perfect match is found (Saiki et al. (1986) Nature 324:163); Saiki et al (1989) Proc. Natl Acad. Sci USA 86:6230). Such allele specific oligonucleotide hybridization techniques may be used to test one mutation or polymorphic region per reaction when oligonucleotides are hybridized to PCR amplified target DNA or a number of different mutations or polymorphic regions when the oligonucleotides are attached to the hybridizing membrane and hybridized with labelled target DNA.

Alternatively, allele specific amplification technology which depends on selective PCR amplification may be used in conjunction with the instant invention. Oligonucleotides used as primers for specific amplification may carry the mutation or polymorphic region of interest in the center of the molecule (so that amplification depends on differential hybridization) (Gibbs et al (1989) Nucleic Acids Res. 17:2437-2448) or at the extreme 3′ end of one primer where, under appropriate conditions, mismatch can prevent, or reduce polymerase extension (Prossner (1993) Tibtech 11:238. In addition it may be desirable to introduce a novel restriction site in the region of the mutation to create cleavage-based detection (Gasparini et al (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

In another embodiment, identification of the allelic variant is carried out using an oligonucleotide ligation assay (OLA), as described, e.g., in U.S. Pat. No. 4,998,617 and in Landegren, U. et al. ((1988) Science 241:1077-1080). The OLA protocol uses two oligonucleotides which are designed to be capable of hybridizing to abutting sequences of a single strand of a target. One of the oligonucleotides is linked to a separation marker, e.g,. biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al. (1990) Proc. Natl. Acad. Sci. USA 87:8923-27). In this method, PCR is used to achieve the exponential amplification of target DNA, which is then detected using OLA.

Several techniques based on this OLA method have been developed and can be used to detect alleles of an IL-1 locus haplotype. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. ((1996) Nucleic Acids Res 24: 3728), OLA combined with PCR permits typing of two alleles in a single microtiter well. By marking each of the allele-specific primers with a unique hapten, i.e. digoxigenin and fluorescein, each OLA reaction can be detected by using hapten specific antibodies that are labeled with different enzyme reporters, alkaline phosphatase or horseradish peroxidase. This system permits the detection of the two alleles using a high throughput format that leads to the production of two different colors.

Another embodiment of the invention is directed to kits for detecting a predisposition for developing a EOA. This kit may contain one or more oligonucleotides, including 5′ and 3′ oligonucleotides that hybridize 5′ and 3′ to at least one allele of an IL-1 locus haplotype. PCR amplification oligonucleotides should hybridize between 25 and 2500 base pairs apart, preferably between about 100 and about 500 bases apart, in order to produce a PCR product of convenient size for subsequent analysis.

Particularly preferred primer pairs for use in the diagnostic method of the invention include the following:

(SEQ ID No. 1)

5′ ATG GTT TTA GAA ATC ATC AAG CCT AGG GCA 3′ and

(SEQ ID No. 2)

5′ AAT GAA AGG AGG GGA GGA TGA CAG AAA TGT 3′;

(SEQ ID No. 3)

5′ TGG CAT TGA TCT GGT TCA TC-3′ and

(SEQ ID No. 4)

5′ GTT TAG GAA TCT TCC CAC TT-3′;

(SEQ ID No. 5)

5′ CTC AGG TGT CCT CGA AGA AAT CAA A 3′ and

(SEQ ID No. 6)

5′ GCT TTT TTG CTG TGA GTC CCG 3′;

(SEQ ID NO. 7)

5′-CTC.AGC.AAC.ACT.CCT.AT-3′ and

(SEQ ID NO. 8)

5′-TCC.TGG.TCT.GCA.GCT.AA-3′;

(SEQ ID NO. 9)

5′-CTA TCT GAG GAA CAA ACT AGT AGC-3′ and

(SEQ ID NO. 10)

5′-TAG GAC ATT GCA CCT AGG GTT TGT-3′;

(SEQ. ID No. 11)

5′ ATT TTT TTA TAA ATC ATC AAG CCT AGG GCA 3′ and

(SEQ. ID No. 12)

5′ AAT TAA AGG AGG GAA GAA TGA CAG AAA TGT 3′;

(SEQ ID NO. 13)

5′-AAG CTT GTT CTA CCA CCT GAA CTA GGC.-3′ and

(SEQ ID NO. 14)

5′-TTA CAT ATG AGC CTT CCA TG.-3′.

The design of additional oligonucleotides for use in the amplification and detection of IL-1 polymorphic alleles by the method of the invention is facilitated by the availability of both updated sequence information from human chromosome 2q13—which contains the human IL-1 locus, and updated human polymorphism information available for this locus. For example, the DNA sequence for the IL-1A, IL-1B and IL-1RN is shown in FIGS. 1 (GenBank Accession No. X03833), 2 (GenBank Accession No. X04500) and 3 (GenBank Accession No. X64532) respectively. Suitable primers for the detection of a human polymorphism in these genes can be readily designed using this sequence information and standard techniques known in the art for the design and optimization of primers sequences. Optimal design of such primer sequences can be achieved, for example, by the use of commercially available primer selection programs such as Primer 2.1, Primer 3 or GeneFisher (See also, Nicklin M. H. J., Weith A. Duff G. W., “A Physical Map of the Region Encompassing the Human Interleukin-1, interleukin-1, and Interleukin-1 Receptor Antagonist Genes” Genomics 19: 382 (1995); Nothwang H. G., et al. “Molecular Cloning of the Interleukin-l gene Cluster: Construction of an Integrated YAC/PAC Contig and a partial transcriptional Map in the Region of Chromosome 2q13” Genomics 41: 370 (1997); Clark, et al. (1986) Nucl. Acids. Res., 14:7897-7914 [published erratum appears in Nucleic Acids Res., 15:868 (1987) and the Genome Database (GDB) project at the URL http://www.gdb.org).

For use in a kit, oligonucleotides may be any of a variety of natural and/or synthetic compositions such as synthetic oligonucleotides, restriction fragments, cDNAs, synthetic peptide nucleic acids (PNAs), and the like. The assay kit and method may also employ labeled oligonucleotides to allow ease of identification in the assays. Examples of labels which may be employed include radio-labels, enzymes, fluorescent compounds, streptavidin, avidin, biotin, magnetic moieties, metal binding moieties, antigen or antibody moieties, and the like.

The kit may, optionally, also include DNA sampling means. DNA sampling means are well known to one of skill in the art and can include, but not be limited to substrates, such as filter papers, the AmpliCard™ (University of Sheffield, Sheffield, England S10 2JF; Tarlow, JW, et al., J. of Invest. Dermatol. 103:387-389 (1994)) and the like; DNA purification reagents such as Nucleon™ kits, lysis buffers, proteinase solutions and the like; PCR reagents, such as 10×reaction buffers, thermostable polymerase, dNTPs, and the like; and allele detection means such as the HinfI restriction enzyme, allele specific oligonucleotides, degenerate oligonucleotide primers for nested PCR from dried blood.

Age-Related Therapeutics and Pharmacogenomics,

Pharmacogenomics

The ability to rapidly genotype patients promises to fundamentally change the testing and development of therapeutic or disease-preventative substances. Currently, the effectiveness of a substance for treating or preventing a disease is assessed by testing it on a pool of patients. While many variables in the patient pool are controlled for, the effects of genetic variability are not typically tested. Consequently, a drug may be found to be statistically ineffective when examined in a genetically diverse pool of patients and yet be highly effective for a select group of patients with particular genetic characteristics. Unless patients are separated by genotype, many drugs with great promise for selected populations are likely to be rejected as useless for the population as a whole.

Knowledge of particular alleles associated with EOA, alone or in conjunction with information on other genetic defects contributing to EOA (the genetic profile of EOA) allows a customization of the therapy to the individual's genetic profile, the goal of “pharmacogenomics”. For example, as shown herein, subjects having an allele associated with EOA, such as IL-1RN (+2018) allele 2 are predisposed to EOA. Thus, comparison of a subject's IL-1 profile to the population profile for the disease, permits the selection or design of drugs that are expected to be safe and efficacious for a particular patient or patient population (i.e., a group of patients having the same genetic alteration).

The ability to target populations expected to show the highest clinical benefit, based on the IL-1 gene profile or the genetic profile of EOA, can enable: 1) the repositioning of marketed drugs with disappointing market results; 2) the rescue of drug candidates whose clinical development has been discontinued as a result of safety or efficacy limitations, which are patient subgroup-specific; and 3) an accelerated and less costly development for drug candidates and more optimal drug labeling (e.g. since measuring the effect of various doses of an agent on an EOA causative mutation is useful for optimizing effective dose).

In one embodiment, a subject's IL-1 genotype and EOA predisposition may be used to tailor a recommended lifestyle, including, for example, changes in exercise and diet. The IL-1 genotype may also be used to recommend nutraceuticals that are predicted to benefit a subject having a particular IL-1 genotype and EOA predisposition.

In another embodiment, subject genotypes and EOA predispositions may be used to manage costs of therapy, by separating patients into groups that are likely or unlikely to benefit from one or more therapeutic regimen. Decisions about the appropriate therapeutic regimen for a subject may be made in view of that subject's grouping, and such procedures may decrease the numbers of patients receiving an unnecessary, ineffective or inappropriate therapeutic regimen. Patients may be separated solely on the basis of genotype or on the basis of genotype in combination with other forms of information, such as lifestyle, age, body-mass index, clinical history, other risk factors, etc. Patients may be sorted into more than one group.

IL-1 Production and Molecular Signaling Pathways

To better understand likely targets for therapeutic intervention and likely EOA biomarkers, it is necessary to understand general mechanisms for IL-1 signaling and production. IL-1 is part of a complex web of inter- and intra-cellular signaling events. Many proteins are involved in the inflammatory response and also in immune responses more generally. A partial list includes the interleukins, TNF, NF-κB, the immunoglobulins, clotting factors, lipoxygenases, as well as attendant receptors, antagonists and processing enzymes for the above.

The IL-1 polypeptides, IL-1α and IL-1β, are abundantly produced by activated macrophages that have been stimulated with bacterial lipopolysaccharide (LPS), TNF, IL-1 itself, other macrophage-derived cytokines, or contact with CD4⁺ T cells. The IL-1 promoter contains several regulatory elements including a cAMP responsive element, an AP-1 binding site and an NF-kB binding site. Both and AP-1 (Jun and Fos) must be activated and translocated to the nucleus in order to regulate transcription. NF-kB is normally retained in the cytoplasm through binding with IkB. The NF-kB-IkB complex is disrupted by phosphorylation of IkB. IkB phosphorylation can be regulated by signaling from cell-surface receptors via activation of mitogen-activated protein kinase (MAP kinse) pathways and other kinase pathways. Jun and Fos are also substrates for regulatory kinases, such as JNK, in the case of Jun.

The IL-1A and B transcripts are translated into pro-proteins by a process that may also be regulated by MAP kinase pathways. Inhibitors of MAP kinase phosphorylation such as trebufelone decrease translation of IL-1 transcripts. The IL-1 α and β precursor proteins require myristoylation for localization to the membrane and conversion to mature IL-1 by the Interleukin Converting Enzyme (ICE), or Caspose I. Other extracellular proteases may also play a minor role in IL-1 maturation, including trypsin, elastase, chymotrypsin and mast cell chymase. ICE can be inhibited by several agents including the eICE isoform, antibodies to the ICE α, β and γ isoforms, the cow pox-produced Crm-A protein and an endogenous tetrapeptide competitive inhibitor.

Mature IL-1α and IL-1β have similar activities and interact with the same receptors. The primary receptor for these factors is the type I IL-1 receptor. The active signaling complex consists of the IL-1 ligand, the type I receptor and the IL-1 receptor accessory protein. A type II receptor, as well as soluble forms of the type I and type II receptors appear to act as decoy receptors to compete for bioavailable IL-1. In addition, a natural inhibitor of IL-1 signaling, IL-1 receptor antagonist, is produced by monocytes. IL-1ra is also produced by hepatocytes and is a major component of the acute phase proteins produced in the liver and secreted into the circulation to regulate immune and inflammatory responses.

The IL-1 signaling complex activates several intracellular signal transduction pathways, including the activities of NF-κB and AP-1 described above. In signaling, IL-1 influences the activity of a host of factors including: PI-3 kinase, phospholipase A2, protein kinase C, the JNK pathway, 5-lipoxygenase, cyclooxygenase 2, p38 MAP kinase, p42/44 MAP kinase, p54 MAP kinase, Rac, Ras, TRAF-6, TRAF-2 and many others. IL-1 also affects expression of a large number of genes including: members of the IL-1 gene cluster, TNF, other interleukin genes (2, 3, 6, 8, 12, 2R, 3R and SR), TGF-β, fibrinogen, matrix metalloprotease 1, collagen, elastase, leukemia inhibiting factor, IFN α, β, γ, COX-2, inducible nitric oxide synthase, metallothioneins, and many more.

EOA Associated Biomarkers

In addition to having genetic tests for EOA, it would be desirable to have tests for monitoring a subject's progression towards or during EOA. In other words, certain biomarkers may be indicative of the timing and/or progression of early onset of aging-related conditions. It would be desirable to be able to identify these biomarkers and monitor them to provide information about the onset and progression of aging-related conditions. It is particularly desirable to find biomarkers that are tailored to the subject's genotype.

In a preferred embodiment, biomarkers likely to be associated with EOA can be identified by using subjects or cells comprising differing IL-1 genotypes. A set of biomarkers can be examined in a subject or cell having an EOA-associated allele, such as IL-1RN (VNTR) allele 2 or another allele of the IL-1 (44112332) or (33221461) haplotype. The same set of biomarkers can be examined in another subject or cell not having an EOA-associated allele. Biomarkers that show a difference dependent upon the IL-1 genotype are likely to be useful for predicting EOA. These differences constitute EOA-associated phenotypes.

The association between certain biomarkers and EOA can be further established by performing trials wherein certain biomarkers are measured in a population of subjects of various ages, some of which may have already begun to evince aging-related conditions. Optionally, multiple measurements may be done over time as subjects age. Preferably, the presence or absence of EOA-associated alleles is determined in the subjects. Standard statistical methods may be used to determine the correlation between certain biomarkers and the early onset of aging-related conditions.

Measurements of EOA-associated biomarkers may be used as an indicator of a subject's current risk of developing EOA or as an indicator of progression towards or through the aging process.

With respect to cells, biomarkers may be essentially any aspect of cell function, for example: levels or rate of production of signaling molecules, transcription factors, intermediate metabolites, cytokines, prostanoids, steroid hormones (eg. estrogen, progesterone, androstenedione or testosterone), gonadotropins (eg. LH and FSH), gene transcripts, post-translational modifications of proteins, gonadotropin releasing hormone (GnRH), catecholamines (eg. dopamine or norepinephrine), opioids, activin, inhibin, as well as IL-1 bioactivities. Biomarkers may include whole genome analysis of transcript levels or whole protEOAe analysis of protein levels and/or modifications. Additionally, biomarkers may be reporter genes. For example, an IL-1 promoter or an IL-1 promoter comprising an EOA-associated allele can be operationally linked to a reporter gene. In an alternative method, the promoter can be an IL-1-regulated promoter, such as IL-8. In this manner, the activity of the reporter gene is reflective of the activity of the promoter. Suitable reporter genes include luciferase (luc), GUS, LacZ, green fluorescent protein (GFP) (and variants thereof, such as RFP, CFP, YFP and BFP), or essentially any other gene that is easily detected. In subjects, biomarkers can be, for example, any of the above as well as electrocardiogram parameters, pulmonary function, IL-6 activities, urine parameters or tissue parameters. Other preferred biomarkers include factors involved in immune and inflammatory responses, as well as factors involved in IL-1 production and signaling, as described above.

EOA Therapeutics

An EOA therapeutic can comprise any type of compound, including a protein, peptide, peptidomimetic, small molecule, nucleic acid, or nutraceutical. In preferred embodiments, an EOA therapeutic is a modulator of a factor involved in IL-1 production or signaling. In a particularly preferred embodiment, an EOA therapeutic is a modulator of IL-1 bioactivity (e.g. IL-1α, IL-1β or an IL-1 receptor agonist or antagonist). Preferred agonists include nucleic acids (e.g. encoding an IL-1 protein or a gene that is up- or down-regulated by an IL-1 protein), protein (e.g. IL-1 proteins or a protein that is up- or down-regulated by an IL-1 protein) or a small molecule (e.g. that regulates expression of an IL-1 protein). Preferred antagonists, which can be identified, for example, using the assays described herein, include nucleic acids (e.g. single (antisense) or double stranded (triplex) DNA or PNA and ribozymes), protein (e.g. antibodies) and small molecules or nutraceuticals that act to suppress or inhibit IL-1 transcription and/or IL-1 activity.

In vivo and Cell-Based Screening Assays

Based on the identification of IL-1 mutations that cause or contribute to EOA, the invention further features in vivo and cell-based assays, e.g., for identifying EOA therapeutics. In one embodiment, a cell having an EOA-associated allele is contacted with a test compound and at least one biomarker is measured. If at least one biomarker changes such that the phenotype of the cell now more closely resembles that of a cell that does not have an EOA-associated allele, then the test substance is likely to be effective as an EOA therapeutic.

As an illustrative example, suppose that an IL-1 allele associated with EOA causes cells having that allele to overproduce an IL-1 polypeptide. Levels of the IL-1 polypeptide are used as a biomarker in this case. Treatment with a test substance causes the cells to produce the IL-1 polypeptide at a lower level, more closely resembling IL-1 polypeptide production in a cell that does not have an EOA-associated allele. Accordingly, the test substance is likely to be effective as an EOA therapeutic. In this manner, test substances with allele-specific effects may be identified. The specificity of the compound vis a vis the IL-1 signaling pathway can, if desired, be confirmed by various control analysis, e.g., measuring the expression of one or more control genes. In particular, this assay can be used to determine the efficacy of IL-1 antisense, ribozyme and triplex compounds.

In another variation a cell is contacted with a test compound and an IL-1 protein and the interaction between the test compound and the IL-1 receptor or between the IL-1 protein (preferably a tagged IL-1 protein) and the IL-1 receptor is detected, e.g., by using a microphysiometer (McConnell et al. (1992) Science 257:1906). An interaction between the IL-1 receptor and either the test compound or the IL-1 protein is detected by the microphysiometer as a change in the acidification of the medium. This assay system thus provides a means of identifying molecular antagonists which, for example, function by interfering with IL-1 protein-IL-1 receptor interactions, as well as molecular agonist which, for example, function by activating an IL-1 receptor.

Essentially any culturable cell type can be used for the cell-based assays. In particular, cells may be immune cells such as monocytes, macrophages or thymocytes, or other cell types such as fibroblasts or cells derived from female reproductive organs. Preferrably cells will express an IL-1 receptor.

In another variation, a subject having an EOA-associated allele is contacted with a test compound and at least one biomarker is measured. If at least one biomarker changes such that the phenotype of the cell now more closely resembles that of a cell that does not have an EOA-associated allele, then the test substance is likely to be effective as an EOA therapeutic. The subject may be a human or a trans genic non-human animal.

In preferred embodiments, cellular or in vivo assays are used to identify compounds which modulate expression of an IL-1 gene, modulate translation of an IL-1 mRNA, or which modulate the stability or activity of an IL-1 mRNA or protein. Accordingly, in one embodiment, a cell which is capable of producing IL-1 protein is incubated with a test compound and the amount of IL-1 protein produced in the cell medium is measured and compared to that produced from a cell which has not been contacted with the test compound. In another variation, an IL-1 bioactivity is measured and compared to the bioactivity measured in a cell which has not been contacted with a test compound. Additionally, the effects of test substances on different cells containing various IL-1 alleles may be compared.

Cell-Free Assays

Cell-free assays can also be used to identify compounds which are capable of interacting with an IL-1 protein, to thereby modify the activity of the IL-1 protein. Such a compound can, e.g., modify the structure of an IL-1 protein thereby affecting its ability to bind to an IL-1 receptor. In a preferred embodiment, cell-free assays for identifying such compounds consist essentially in a reaction mixture containing an IL-1 protein and a test compound or a library of test compounds in the presence or absence of a binding partner. A test compound can be, e.g., a derivative of an IL-1 binding partner, e.g., a biologically inactive target peptide, or a small molecule.

Accordingly, one exemplary screening assay of the present invention includes the steps of contacting an IL-1 protein or functional fragment thereof with a test compound or library of test compounds and detecting the formation of complexes. For detection purposes, the molecule can be labeled with a specific marker and the test compound or library of test compounds labeled with a different marker. Interaction of a test compound with an IL-1 protein or fragment thereof can then be detected by determining the level of the two labels after an incubation step and a washing step. The presence of two labels after the washing step is indicative of an interaction.

An interaction between molecules can also be identified by using real-time BIA (Biomolecular Interaction Analysis, Pharmacia Biosensor AB) which detects surface plasmon resonance (SPR), an optical phenomenon. Detection depends on changes in the mass concentration of macromolecules at the biospecific interface, and does not require any labeling of interactants. In one embodiment, a library of test compounds can be immobilized on a sensor surface, e.g., which forms one wall of a micro-flow cell. A solution containing the IL-1β protein or functional fragment thereof is then flown continuously over the sensor surface. A change in the resonance angle as shown on a signal recording, indicates that an interaction has occurred. This technique is further described, e.g., in BlAtechnology Handbook by Pharmacia.

Another exemplary screening assay of the present invention includes the steps of (a) forming a reaction mixture including: (i) an IL-1 protein, (ii) an IL-1 receptor, and (iii) a test compound; and (b) detecting interaction of the IL-1 protein and IL-1 receptor. A statistically significant change (potentiation or inhibition) in the interaction of the IL-1 protein and IL-1 receptor in the presence of the test compound, relative to the interaction in the absence of the test compound, indicates a potential antagonist (inhibitor) of IL-1 bioactivity for the test compound. The compounds of this assay can be contacted simultaneously. Alternatively, an IL-1 protein can first be contacted with a test compound for an appropriate amount of time, following which the IL-1β receptor is added to the reaction mixture. The efficacy of the compound can be assessed by generating dose response curves from data obtained using various concentrations of the test compound. Moreover, a control assay can also be performed to provide a baseline for comparison.

Complex formation between an IL-1 protein and IL-1 receptor may be detected by a variety of techniques. Modulation of the formation of complexes can be quantitated using, for example, detectably labeled proteins such as radiolabeled, fluorescently labeled, or enzymatically labeled IL-1 protein or IL-1 receptors, by immunoassay, or by chromatographic detection.

Typically, it will be desirable to immobilize either IL-1 protein or the IL-1 receptor to facilitate separation of complexes from uncomplexed forms of one or both of the proteins, as well as to accommodate automation of the assay. Binding of IL-1 protein and IL-1 receptor can be accomplished in any vessel suitable for containing the reactants. Examples include microtitre plates, test tubes, and micro-centrifuge tubes. In one embodiment, a fusion protein can be provided which adds a domain that allows the protein to be bound to a matrix. For example, glutathione-S-transferase/IL-1β (GST/IL-1β) fusion proteins can be adsorbed onto glutathione sepharose beads (Sigma Chemical, St. Louis, Mo.) or glutathione derivatized microtitre plates, which are then combined with the IL-1 receptor, e.g. an 35S-labeled IL-1 receptor, and the test compound, and the mixture incubated under conditions conducive to complex formation, e.g. at physiological conditions for salt and pH, though slightly more stringent conditions may be desired. Following incubation, the beads are washed to remove any unbound label, and the matrix immobilized and radiolabel determined directly (e.g. beads placed in scintilant), or in the supernatant after the complexes are subsequently dissociated. Alternatively, the complexes can be dissociated from the matrix, separated by SDS-PAGE, and the level of IL-1 protein or IL-1 receptor found in the bead fraction quantitated from the gel using standard electrophoretic techniques such as described in the appended examples. Other techniques for immobilizing proteins on matrices are also available for use in the subject assay. For instance, either IL-1 protein or IL-1 receptor can be immobilized utilizing conjugation of biotin and streptavidin.

Transgenic Animals

As described above, transgenic animals can be made for example, to assist in screening for EOA therapeutics. Transgenic animals of the invention can include non-human animals containing an IL-1 mutation, which is causative of EOA in humans, under the control of an appropriate IL-1 promoter or under the control of a heterologous promoter. Transgenic animals of the invention can also include an IL-1 gene expressed at such a level as to create an EOA phenotype. To compare the effects of different IL-1 alleles, transgenic animals may be generated with a variety of IL-1 alleles and differences in EOA phenotype can be identified. By testing different alleles and different expression levels, an animal with an EOA phenotype optimal for testing candidate drugs can be generated and identified.

The transgenic animals can also be animals containing a transgene, such as reporter gene, under the control of an IL-1 promoter or fragment thereof. These animals are useful, e.g., for identifying drugs that modulate production of an IL-1, such as by modulating gene expression. In certain variations, the IL-1 allele may be a promoter mutation. In this case it is particularly desirable to operationally fuse the altered promoter to a suitable reporter gene.

Methods for obtaining transgenic non-human animals are well known in the art. In preferred embodiments, the expression of the EOA causative mutation is restricted to specific subsets of cells, tissues or developmental stages utilizing, for example, cis-acting sequences that control expression in the desired pattern. In the present invention, such mosaic expression of an IL-1 protein can be essential for many forms of lineage analysis and can additionally provide a means to assess the effects of, for example, expression level which might grossly alter development in small patches of tissue within an otherwise normal embryo. Toward this end, tissue-specific regulatory sequences and conditional regulatory sequences can be used to control expression of the IL-1 mutation in certain spatial patterns. Moreover, temporal patterns of expression can be provided by, for example, conditional recombination systems or prokaryotic transcriptional regulatory sequences. Genetic techniques, which allow for the expression of IL-1 mutation can be regulated via site-specific genetic manipulation in vivo, are known to those skilled in the art.

The transgenic animals of the present invention all include within a plurality of their cells an EOA causative mutation transgene of the present invention, which transgene alters the phenotype of the “host cell”. In an illustrative embodiment, either the cre/loxP recombinase system of bacteriophage P1 (Lakso et al. (1992) PNAS 89:6232-6236; Orban et al. (1992) PNAS 89:6861-6865) or the FLP recombinase system of Saccharomyces cerevisiae (O'Gorman et al. (1991) Science 251:1351-1355; PCT publication WO 92/15694) can be used to generate in vivo site-specific genetic recombination systems. Cre recombinase catalyzes the site-specific recombination of an intervening target sequence located between loxP sequences. loxP sequences are 34 base pair nucleotide repeat sequences to which the Cre recombinase binds and are required for Cre recombinase mediated genetic recombination. The orientation of loxP sequences determines whether the intervening target sequence is excised or inverted when Cre recombinase is present (Abremski et al. (1984) J. Biol. Chem. 259:1509-1514); catalyzing the excision of the target sequence when the loxP sequences are oriented as direct repeats and catalyzes inversion of the target sequence when loxP sequences are oriented as inverted repeats.

Accordingly, genetic recombination of the target sequence is dependent on expression of the Cre recombinase. Expression of the recombinase can be regulated by promoter elements which are subject to regulatory control, e.g., tissue-specific, developmental stage-specific, inducible or repressible by externally added agents. This regulated control will result in genetic recombination of the target sequence only in cells where recombinase expression is mediated by the promoter element. Thus, the activation of expression of the EOA causative mutation transgene can be regulated via control of recombinase expression.

Use of the cre/loxP recombinase system to regulate expression of an EOA causative mutationa transgene requires the construction of a transgenic animal containing transgenes encoding both the Cre recombinase and the subject protein. Animals containing both the Cre recombinase and the EOA causative mutation transgene can be provided through the construction of “double” transgenic animals. A convenient method for providing such animals is to mate two transgenic animals each containing a transgene.

Similar conditional transgenes can be provided using prokaryotic promoter sequences which require prokaryotic proteins to be simultaneous expressed in order to facilitate expression of the transgene. Exemplary promoters and the corresponding trans-activating prokaryotic proteins are given in U.S. Pat. No. 4,833,080.

Moreover, expression of the conditional transgenes can be induced by gene therapy-like methods wherein a gene encoding the transactivating protein, e.g. a recombinase or a prokaryotic protein, is delivered to the tissue and caused to be expressed, such as in a cell-type specific manner. By this method, the transgene could remain silent into adulthood until “turned on” by the introduction of the transactivator.

In an exemplary embodiment, the “transgenic non-human animals” of the invention are produced by introducing transgenes into the germline of the non-human animal. Embryonal target cells at various developmental stages can be used to introduce transgenes. Different methods are used depending on the stage of development of the embryonal target cell. The specific line(s) of any animal used to practice this invention are selected for general good health, good embryo yields, good pronuclear visibility in the embryo, and good reproductive fitness. In addition, the haplotype is a significant factor. For example, when transgenic mice are to be produced, strains such as C57BL/6 or FVB lines are often used (Jackson Laboratory, Bar Harbor, Me.). Preferred strains are those with H-2b, H-2d or H-2q haplotypes such as C57BL/6 or DBA/1. The line(s) used to practice this invention may themselves be transgenics, and/or may be knockouts (i.e., obtained from animals which have one or more genes partially or completely suppressed).

In one embodiment, the transgene construct is introduced into a single stage embryo. The zygote is the best target for microinjection. In the mouse, the male pronucleus reaches the size of approximately 20 micrometers in diameter which allows reproducible injection of 1-2 pl of DNA solution. The use of zygotes as a target for gene transfer has a major advantage in that in most cases the injected DNA will be incorporated into the host gene before the first cleavage (Brinster et al. (1985) PNAS 82:4438-4442). As a consequence, all cells of the transgenic animal will carry the incorporated transgene. This will in general also be reflected in the efficient transmission of the transgene to offspring of the founder since 50% of the germ cells will harbor the transgene.

Normally, fertilized embryos are incubated in suitable media until the pronuclei appear. At about this time, the nucleotide sequence comprising the transgene is introduced into the female or male pronucleus as described below. In some species such as mice, the male pronucleus is preferred. It is most preferred that the exogenous genetic material be added to the male DNA complement of the zygote prior to its being processed by the ovum nucleus or the zygote female pronucleus. It is thought that the ovum nucleus or female pronucleus release molecules which affect the male DNA complement, perhaps by replacing the protamines of the male DNA with histones, thereby facilitating the combination of the female and male DNA complements to form the diploid zygote.

Thus, it is preferred that the exogenous genetic material be added to the male complement of DNA or any other complement of DNA prior to its being affected by the female pronucleus. For example, the exogenous genetic material is added to the early male pronucleus, as soon as possible after the formation of the male pronucleus, which is when the male and female pronuclei are well separated and both are located close to the cell membrane. Alternatively, the exogenous genetic material could be added to the nucleus of the sperm after it has been induced to undergo decondensation. Sperm containing the exogenous genetic material can then be added to the ovum or the decondensed sperm could be added to the ovum with the transgene constructs being added as soon as possible thereafter.

Introduction of the transgene nucleotide sequence into the embryo may be accomplished by any means known in the art such as, for example, microinjection, electroporation, or lipofection. Following introduction of the transgene nucleotide sequence into the embryo, the embryo may be incubated in vitro for varying amounts of time, or reimplanted into the surrogate host, or both. In vitro incubation to maturity is within the scope of this invention. One common method in to incubate the embryos in vitro for about 1-7 days, depending on the species, and then reimplant them into the surrogate host.

For the purposes of this invention a zygote is essentially the formation of a diploid cell which is capable of developing into a complete organism. Generally, the zygote will be comprised of an egg containing a nucleus formed, either naturally or artificially, by the fusion of two haploid nuclei from a gamete or gametes. Thus, the gamete nuclei must be ones which are naturally compatible, i.e., ones which result in a viable zygote capable of undergoing differentiation and developing into a functioning organism. Generally, a euploid zygote is preferred. If an aneuploid zygote is obtained, then the number of chromosomes should not vary by more than one with respect to the euploid number of the organism from which either gamete originated.

In addition to similar biological considerations, physical ones also govern the amount (e.g., volume) of exogenous genetic material which can be added to the nucleus of the zygote or to the genetic material which forms a part of the zygote nucleus. If no genetic material is removed, then the amount of exogenous genetic material which can be added is limited by the amount which will be absorbed without being physically disruptive. Generally, the volume of exogenous genetic material inserted will not exceed about 10 picoliters. The physical effects of addition must not be so great as to physically destroy the viability of the zygote. The biological limit of the number and variety of DNA sequences will vary depending upon the particular zygote and functions of the exogenous genetic material and will be readily apparent to one skilled in the art, because the genetic material, including the exogenous genetic material, of the resulting zygote must be biologically capable of initiating and maintaining the differentiation and development of the zygote into a functional organism.

The number of copies of the transgene constructs which are added to the zygote is dependent upon the total amount of exogenous genetic material added and will be the amount which enables the genetic transformation to occur. Theoretically only one copy is required; however, generally, numerous copies are utilized, for example, 1,000-20,000 copies of the transgene construct, in order to insure that one copy is functional. As regards the present invention, there will often be an advantage to having more than one functioning copy of each of the inserted exogenous DNA sequences to enhance the phenotypic expression of the exogenous DNA sequences.

Any technique which allows for the addition of the exogenous genetic material into nucleic genetic material can be utilized so long as it is not destructive to the cell, nuclear membrane or other existing cellular or genetic structures. The exogenous genetic material is preferentially inserted into the nucleic genetic material by microinjection. Microinjection of cells and cellular structures is known and is used in the art.

Reimplantation is accomplished using standard methods. Usually, the surrogate host is anesthetized, and the embryos are inserted into the oviduct. The number of embryos implanted into a particular host will vary by species, but will usually be comparable to the number of off spring the species naturally produces.

Transgenic offspring of the surrogate host may be screened for the presence and/or expression of the transgene by any suitable method. Screening is often accomplished by Southern blot or Northern blot analysis, using a probe that is complementary to at least a portion of the transgene. Western blot analysis using an antibody against the protein encoded by the transgene may be employed as an alternative or additional method for screening for the presence of the transgene product. Typically, DNA is prepared from tail tissue and analyzed by Southern analysis or PCR for the transgene. Alternatively, the tissues or cells believed to express the transgene at the highest levels are tested for the presence and expression of the transgene using Southern analysis or PCR, although any tissues or cell types may be used for this analysis.

Alternative or additional methods for evaluating the presence of the transgene include, without limitation, suitable biochemical assays such as enzyme and/or immunological assays, histological stains for particular marker or enzyme activities, flow cytometric analysis, and the like. Analysis of the blood may also be useful to detect the presence of the transgene product in the blood, as well as to evaluate the effect of the transgene on the levels of various types of blood cells and other blood constituents.

Progeny of the transgenic animals may be obtained by mating the transgenic animal with a suitable partner, or by in vitro fertilization of eggs and/or sperm obtained from the transgenic animal. Where mating with a partner is to be performed, the partner may or may not be transgenic and/or a knockout; where it is transgenic, it may contain the same or a different transgene, or both. Alternatively, the partner may be a parental line. Where in vitro fertilization is used, the fertilized embryo may be implanted into a surrogate host or incubated in vitro, or both. Using either method, the progeny may be evaluated for the presence of the transgene using methods described above, or other appropriate methods.

The transgenic animals produced in accordance with the present invention will include exogenous genetic material. Further, in such embodiments the sequence will be attached to a transcriptional control element, e.g., a promoter, which preferably allows the expression of the transgene product in a specific type of cell.

Retroviral infection can also be used to introduce the transgene into a non-human animal. The developing non-human embryo can be cultured in vitro to the blastocyst stage. During this time, the blastomeres can be targets for retroviral infection (Jaenich, R. (1976) PNAS 73:1260-1264). Efficient infection of the blastomeres is obtained by enzymatic treatment to remove the zona pellucida (Manipulating the Mouse Embryo, Hogan eds. (Cold Spring Harbor Laboratory Press, Cold Spring Harbor, 1986). The viral vector system used to introduce the transgene is typically a replication-defective retrovirus carrying the transgene (Jahner et al. (1985) PNAS 82:6927-6931; Van der Putten et al. (1985) PNAS 82:6148-6152). Transfection is easily and efficiently obtained by culturing the blastomeres on a monolayer of virus-producing cells (Van der Putten, supra; Stewart et al. (1987) EMBO J. 6:383-388). Alternatively, infection can be performed at a later stage. Virus or virus-producing cells can be injected into the blastocoele (Jahner et al. (1982) Nature 298:623-628). Most of the founders will be mosaic for the transgene since in corporation occurs only in a subset of the cells which formed the transgenic non-human animal. Further, the founder may contain various retroviral insertions of the transgene at different positions in the genome which generally will segregate in the offspring. In addition, it is also possible to introduce transgenes into the germ line by intrauterine retroviral infection of the midgestation embryo (Jahner et al. (1982) supra).

A third type of target cell for transgene introduction is the embryonal stem cell (ES). ES cells are obtained from pre-implantation embryos cultured in vitro and fused with embryos (Evans et al. (1981) Nature 292:154-156; Bradley et al. (1984) Nature 309:255-258; Gossler et al. (1986) PNAS 83: 9065-9069; and Robertson et al. (1986) Nature 322:445-448). Transgenes can be efficiently introduced into the ES cells by DNA transfection or by retrovirus-mediated transduction. Such transformed ES cells can thereafter be combined with blastocysts from a non-human animal. The ES cells thereafter colonize the embryo and contribute to the germ line of the resulting chimeric animal. For review see Jaenisch, R. (1988) Science 240:1468-1474.

Effective Dose

Toxicity and therapeutic efficacy of such compounds can be determined by standard pharmaceutical procedures in cell cultures or experimental animals, e.g., for determining The Ld50 (The Dose Lethal To 50% Of The Population) And The Ed50 (the dose therapeutically effective in 50% of the population). The dose ratio between toxic and therapeutic effects is the therapeutic index and it can be expressed as the ratio LD50/ED50. Compounds which exhibit large therapeutic induces are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Formulation and Use

Pharmaceutical compositions for use in accordance with the present invention may be formulated in conventional manner using one or more physiologically acceptable carriers or excipients. Thus, the compounds and their physiologically acceptable salts and solvates may be formulated for administration by, for example, injection, inhalation or insufflation (either through the mouth or the nose) or oral, buccal, parenteral or rectal administration.

For such therapy, the compounds of the invention can be formulated for a variety of loads of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, and subcutaneous. For injection, the compounds of the invention can be formulated in liquid solutions, preferably in physiologically compatible buffers such as Hank's solution or Ringer's solution. In addition, the compounds may be formulated in solid form and redissolved or suspended immediately prior to use. Lyophilized forms are also included.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example Osubcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. in addition, detergents may be used to facilitate permeation. Transmucosal administration may be through nasal sprays or using suppositories. For topical administration, the oligomers of the invention are formulated into ointments, salves, gels, or creams as generally known in the art. A wash solution can be used locally to treat an injury or inflammation to accelerate healing.

In clinical settings, a gene delivery system for the EOA therapeutic gene can be introduced into a patient by any of a number of methods, each of which is familiar in the art. For instance, a pharmaceutical preparation of the gene delivery system can be introduced systemically, e.g., by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by stereotactic injection (e.g., Chen et al. (1994) PNAS 91: 3054-3057). An EOA therapeutic gene can be delivered in a gene therapy construct by electroporation using techniques described, for example, by Dev et al. ((1994) Cancer Treat Rev 20:105-115).

The pharmaceutical preparation of the gene therapy construct or compound of the inventioncan consist essentially of the gene delivery system in an acceptable diluent, or can comprise a slow release matrix in which the gene delivery vehicle or compound is imbedded. Alternatively, where the complete gene delivery system can be produced intact from recombinant cells, e.g., retroviral vectors, the pharmaceutical preparation can comprise one or more cells which produce the gene delivery system.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

### EXAMPLES

The invention now being generally described, it will be more readily understood by reference to the following examples, which are included merely for purposes of illustration of certain aspects and embodiments of the present invention, and are not intended to limit the invention.

### Example 1

### Association Between Aging and Alleles

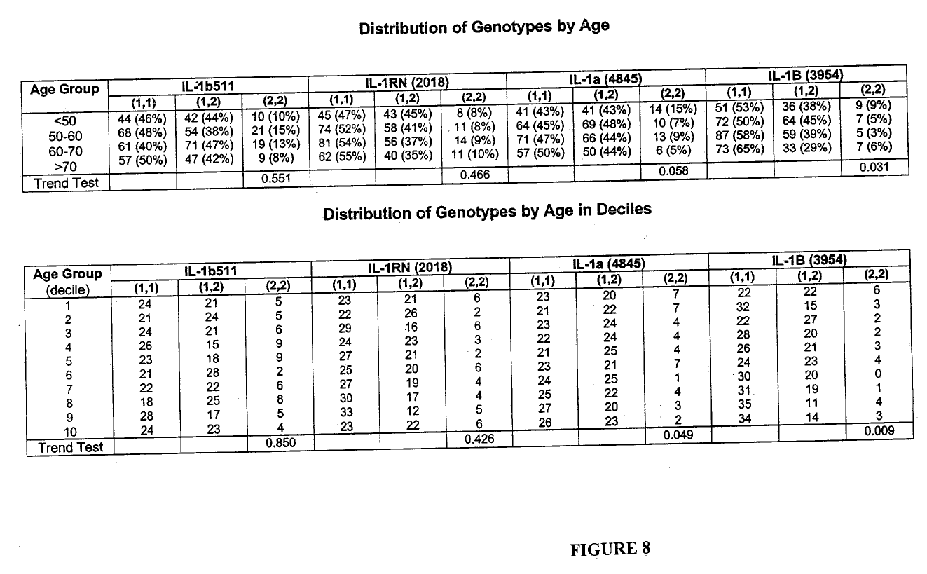

In studies at the Mayo clinic, involving 502 adult Caucasians, carriage of specific genotypes was found to decline significantly with age, suggesting a selective loss of these genotypes due to age-related diseases (See FIGS. 1 and 8).

Carriage of allele 2 declined with age for IL-1A(+4845) and IL-1B(+3954), both of which are in strong linkage disequilibrium and are characteristic of what we refer to as Pattern 1.

Carriage of allele 2 at IL-1RN(+2018) also shows a decrease with age. This marker is in negative linkage disequilibrium with both IL-1A(+4845) and IL-1B(+3954) and is characteristic of Pattern 2. Therefore the decrease in the RN(+2018) allele 2 is most likely not merely reflecting the Pattern 1 effects, but this seems to suggest an independent relationship between IL-1 RN(+2018) (and Pattern 2) and aging.

### Example 2

### IL-1RN Allele 2 is Associated with Endothelial Cell Senescence

IL-1 genotype predicts the length of the endothelial cell life span by influencing the senescence program in that cell type. IL-1 genotype patterns may be used to predict senescence in a wide variety of cell types and may play a role in the progression of numerous immunoinflammatory diseases.

Study Design:

Human umbilical vein endothelial cells (HUVEC) were isolated and maintained in culture. The number of population doublings were assessed in these cells and correlated with the IL-1RN VNTR polymorphism. The cells were derived from Northern European Caucasian donors. Of the donor HUVEC populations tested, 11 carried IL-1RN VNTR 1.1, 8 carried IL-1RN VNTR 1.2 and 2 carried IL-1RN VNTR 2.2. The hypothesis to be tested was the association with the rare IL-1VNTR allele 2 with decreased cell population doublings, which is an indicator of senescence.

Results:

A significant association was found between the IL-1ra genotype and the number of cumulative population doublings in culture. The presence of the 2 allele of the VNTR polymorphism is associated with a reduction in the proliferative capacity of HUVEC in culture. This affect is most marked in the homozygous state but the presence of any 2 allele is associated with a significant reduction in replicative capacity (p=0.0448). The IL-1RN (VNTR) allele 2 is a part of Pattern 2. While not wishing to be limited to any particular mechanism, it is possible that the IL-1 genotype affects HUVEC senescence through an effect on nitric oxide levels. The IL-1 family of cytokines is known to modulate nitric oxide levels, and nitric oxide appears to delay endothelial cell senescence in culture, in part by delaying the age-dependent decrease in telomerase activity.

### Example 3

### Exemplary Methods for Detection of Certain IL-1 Alleles

Blood is taken by venipuncture and stored uncoagulated at −20° C. prior to DNA extraction. Ten milliliters of blood are added to 40 ml of hypotonic red blood cell (RBC) lysis solution (10 mM Tris, 0.32 Sucrose, 4 mM MgCl2, 1% Triton X-100) and mixed by inversion for 4 minutes at room temperature (RT). Samples are then centrifuged at 1300 g for 15 minutes, the supernatant aspirated and discarded, and another 30 ml of RBC lysis solution added to the cell pellet. Following centrifugation, the pellet is resuspended in 2 ml white blood cell (WBC) lysis solution (0.4 M Tris, 60 mM EDTA, 0.15 M NaCl, 10% SDS) and transferred into a fresh 15 ml polypropylene tube. Sodium perchlorate is added at a final concentration of 1M and the tubes are first inverted on a rotary mixer for 15 minutes at RT, then incubated at 65° C. for 25 minutes, being inverted periodically. After addition of 2 ml of chloroform (stored at −20° C.), samples are mixed for 10 minutes at room temperature and then centrifuged at 800 G for 3 minutes. At this stage, a very clear distinction of phases can be obtained using 300 1 Nucleon Silica suspension (Scotlab, UK) and centrifugation at 1400 G for 5 minutes. The resulting aqueous upper layer is transferred to a fresh 15 ml polypropylene tube and cold ethanol (stored at −20° C.) is added to precipitate the DNA. This is spooled out on a glass hook and transferred to a 1.5 ml eppendorf tube containing 500 1 TE or sterile water. Following overnight resuspension in TE, genomic DNA yield is calculated by spectrophotometry at 260 nm. Aliquots of samples are diluted at 100 ug/ml, transferred to microtiter containers and stored at 4° C. Stocks are stored at −20° C. for future reference.

Generally, alleles are detected by PCR followed by a restriction digest or hybridization with a probe. Exemplary primer sets and analyses are presented for exemplary loci.

IL-1RN (+2018). PCR primers are designed (mismatched to the genomic sequence) to engineer two enzyme cutting sites on the two alleles to allow for RFLP analysis. The gene accesion number is X64532. Oligonucleotide primers are:

5′ CTATCTGAGGAACAACCAACTAGTAGC 3′

5′ TAGGACATTGCACCTAGGGTTTGT 3′

Cycling is performed at [96° C., 1 min]; [94° C., 1 min; 57° C., 1 min; 70° C., 2 min;]×35; [70° C., 5 min]×1; 4° C. Each PCR reaction is divided in two 25 ul aliquots: to one is added 5 Units of Alu 1, to the other 5 Units of Msp 1, in addition to 3 ul of the specific 10× restriction buffer. Incubation is at 37° C. overnight. Electrophoresis is by PAGE 9%.

The two enzymes cut respectively the two different alleles. Alu 1 will produce 126 and 28 bp fragments for allele 1, while it does not digest allele 2 (154 bp). Msp 1 will produce 125 and 29 bp with allele 2, while allele 1 is uncut (154 bp). Hence the two reactions (separated side by side in PAGE) will give inverted patterns of digestion for homozygotes, and identical patterns in heterozygotes. Allelic frequencies are 0.74 and 0.26.

IL-1RN (VNTR). The IL1-RN (VNTR) marker may be genotyped in accordance with the following procedure. As indicated above, the two alleles of the IL1-RN (+2018) marker are >97% in linkage disequilibrium with the two most frequent alleles of IL-1RN (VNTR), which are allele 1 and allele 2. The gene accession number is X64532. The oligonucleotide primers used for PCR amplification are:

5′ CTCAGCAACACTCCTAT 3′

5′ TCCTGGTCTGCAGGTAA 3′

Cycling is performed at [96° C., 1 min]×1; [94° C., 1 min; 60° C., 1 min; 70° C., 2 min]×35; [70° C., 5 min]×1; 4° C. Electrophoresis is conducted in 2% agarose at 90V for 30 min.

The PCR product sizes are direct indication of number of repeats: the most frequent allele (allele 1) yields a 412 bp product. As the flanking regions extend for 66 bp, the remaining 344 bp imply four 86 bp repeats. Similarly, a 240 bp product indicates 2 repeats (allele 2), 326 is for 3 repeats (allele 3), 498 is 5 (allele 4), 584 is 6 (allele 6). Frequencies for the four most frequent alleles are 0.734, 0.241, 0.021 and 0.004.

IL-1A (−889). The IL-1A (−889) marker may be genotyped in accordance with the following procedure. McDowell et al., Arthritis Rheum. 38:221-28, 1995. One of the PCR primers has a base change to create an Nco I site when amplifying allele 1 (C at −889) to allow for RFLP analysis. The gene accession number is X03833. The oligonucleotide primers used for PCR amplification are:

5′ AAG CTT GTT CTA CCA CCT GAA CTA GGC 3′

5′ TTA CAT ATG AGC CTT CCA TG 3′

MgCl2 is used at 1 mM final concentration, and PCR primers are used at 0.8 μM. Cycling is performed at [96° C., 1 min]×1; [94° C., 1 min; 50° C., 1 min; 72° C., 2 min]×45; [72° C., 5 min]×1; 4° C. To each PCR reaction is added 6 Units of Nco I in addition to 3 μl of the specific 10×restriction buffer. Incubation is at 37/overnight. Electrophoresis is conducted by 6% PAGE. Nco I digest will produce fragments 83 and 16 bp in length, whereas the restriction enzyme does not cut allele 2. Correspondingly, heterozygotes will have three bands. Frequencies for the two alleles are 0.71 and 0.29.

IL-1A (+4845). The IL-1A (+4845) marker may be genotyped in accordance with the following procedure. The PCR primers create an Fnu 4H1 restriction site in allele 1 to allow for RFLP analysis. The gene accession number is X03833. The oligonucleotide primers used for PCR amplification are:

5′ ATG GTT TTA GAA ATC ATC AAG CCT AGG GCA 3′

5′ AAT GAA AGG AGG GGA GGA TGA CAG AAA TGT 3′

MgCl2 is used at 1 mM final concentration, and PCR primers are used at 0.8 μM. DMSO is added at 5% and DNA template is at 150 ng/50 μl PCR. Cycling is performed at [95° C., 1 min]×1; [94° C., 1 min; 56° C., 1 min; 72° C., 2 min]×35; [72° C., 5 min]×1; 4° C. To each PCR reaction is added 2.5 Units of Fnu 4H1 in addition to 2 μl of the specific 10×restriction buffer. Incubation is at 37° C. overnight. Electrophoresis is conducted by 9% PAGE.

Fnu 4H1 digest will produce a constant band of 76 bp(present regardless of the allele), and two further bands of 29 and 124 bp for allele 1, and a single further band of 153 bp for allele 2. Frequencies for the two alleles are 0.71 and 0.29.

IL-1B (−511). The IL-1B (−511) marker may be genotyped in accordance with the following procedure. The gene accession number is X04500. The oligonucleotide primers used for PCR amplification are:

5′ TGG CAT TGA TCT GGT TCA TC 3′

5′ GTT TAG GAA TCT TCC CAC TT 3′

MgCl2 is used at 2.5 mM final concentration, and PCR primers are used at 1 PM. Cycling is performed at [95° C., 1 min]×1; [95° C., 1 min; 53° C., 1 min; 72° C., 1 min]×35; [72° C., 5 min]×1; 4° C. Each PCR reaction is divided into two aliquots: to one aliquot is added 3 Units of Ava I, to the other aliquot is added 3.7 Units of Bsu 36I. To both aliquots is added 3 μl of the specific 10×restriction buffer. Incubation is at 37° C. overnight. Electrophoresis is conducted by 9% PAGE.

Each of the two restriction enzymes cuts one of the two alleles, which allows for RFLP analysis. Ava I will produce two fragments of 190 and 114 bp with allele 1, and it does not cut allele 2 (304 bp). Bsu 36I will produce two fragments of 190 and 11 base pairs with allele 2, and it does not cut allele 1 (304 bp). Frequencies for the two alleles are 0.61 and 0.39.

IL-1B (+3954). The IL-1B (+3954) marker may be genotyped in accordance with the following procedure. The gene accession number is X04500. The oligonucleotide primers used for PCR amplification are:

5′ CTC AGG TGT CCT CGA AGA AAT CAA A 3′

5′ GCT TTT TTG CTG TGA GTC CCG 3′

MgCl2 is used at 2.5 mM final concentration, and DNA template at 150 ng/50 μl PCR. Cycling is performed at [95° C., 2 min]×1; [95° C., 1 min; 67.5° C., 1 min; 72° C., 1 min]×35; [72° C., 5 min]×1; 4° C. To each PCR reaction is added 10 Units of Taq I (Promega) in addition to 3 μl of the specific 10×restriction buffer. Incubation is at 65/overnight. Electrophoresis is conducted by 9% PAGE.

The restriction enzyme digest produces a constant band of 12 bp and either two further bands of 85 and 97 bp corresponding to allele 1, or a single band of 182 bp corresponding to allele 2. Frequencies for the two alleles are 0.82 and 0.18.

IL-1B (−3737): Methods for detection of this allele are described in detail in U.S. provisional application No. 60/331,681, filed Nov. 19, 2001.

Incorporation by Reference

All publications and patents mentioned herein are hereby incorporated by reference in their entirety as if each individual publication or patent was specifically and individually indicated to be incorporated by reference. In case of conflict, the present application, including any definitions herein, will control.

Equivalents

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

What is claimed:

1. A method for determining a subject's susceptibility to the early onset or progression of an aging-related condition, comprising accessing the subject's genotype with respect to at least one allele of an IL-1 pattern 1, pattern 2 and/or pattern 3, wherein the presence or absence of the at least one allele of an IL-1 pattern 1, pattern 2 and/or pattern 3 provides information about the subject's susceptibility to an early onset or progression of an aging-related condition.

2. The method of claim 1, wherein accessing a subject's genotype comprises obtaining previously known information about the subject's genotype.

3. The method of claim 1, wherein accessing a subject's genotype comprises:
(a) obtaining a nucleic acid sample from said subject; and
(b) detecting at least one allele of IL-1 pattern 1, pattern 2 and/or pattern 3.

4. The method of claim 1, wherein a subject having an allele of IL-1 pattern 1 or pattern indicates that the subject susceptible to an earlier onset or progression of an aging-related condition.

5. The method of claim 1 wherein the at least one allele of the pattern 1 is selected from the group consisting of: the IL-1A (222/223) allele 4, IL-1A (gz5/gz6) allele 4, IL-1A (−889) allele 1, allele 3 of the gaat.p33330 marker of the IL-1B/IL-1RN intergenic region, allele 3 of the Y31 marker of the IL-1B/IL-1RN intergenic region, IL-1RN (+2018) allele 2, IL-1A (+4845) allele 1, IL-1B (+3954) allele 1, IL-1B (−3737) allele 1, and IL-1RN (VNTR) allele 2.

6. The method of claim 1 wherein the at least one allele of the pattern 2 is selected from the group consisting of: the IL-1A (222/223) allele 3, IL-1A (gz5/gz6) allele 3, IL-1A (−889) allele 2, allele 4 of the gaat.p33330 marker of the IL-1B/IL-1RN intergenic region, allele 6 of the Y31 marker of the IL-1B/IL-1RN intergenic region, IL-1RN (+2018) allele 1, IL-1A (+4845) allele 2, IL-1B (+3954) allele 2, IL-1 B (−3737) allele 1, and IL-1RN (VNTR) allele 1.

7. The method of claim 1 wherein the at least one allele of the pattern 3 is selected from the group consisting of: the IL-1A (−889) allele 1, IL-1A (+4845) allele 1, IL-1B (−3737) allele 2, IL-1B (+3954) allele 1, IL-1RN (+2018) allele 1, and IL-1RN (VNTR) allele 1.

8. A method of claim 1 wherein said at least one allele is selected from the group consisting of IL-1RN (VNTR) allele 2, IL-1RN (+2018) allele 2, IL-1A (+4845) allele 2 and IL-1B (+3954) allele 2.

9. The method of claim 1, wherein the aging related condition is selected from the group consisting of: aging-related conditions of the invention include impaired connective tissue function, cardiovascular disease, age-related cancer, abnormal immune system function and impaired neurological function.

10. A method for selecting an appropriate therapeutic regimen or lifestyle recommendation for a subject, comprising accessing the subject's genotype with respect to at least one allele of an IL-1 pattern 1 or pattern 2 or pattern 3, wherein the presence or absence of the at least one allele of an IL-1 pattern 1, pattern 2 and/or pattern 3 provides information about the subject's susceptibility to an early onset or progression of an aging-related condition, and allowing the selection of a therapeutic regimen or lifestyle recommendation that is suitable to the subject's susceptibility to an early onset of an aging-related condition.

11. A method of claim 9, wherein the therapeutic regimen comprises administering a nutraceutical.

12. The method of claim 9, wherein accessing a subject's genotype comprises obtaining previously known information about the subject's genotype.

13. The method of claim 9, wherein accessing a subject's genotype comprises:
(a) obtaining a nucleic acid sample from said subject; and
(b) detecting at least one allele of IL-1 pattern 1, pattern 2 and/or pattern 3.

14. The method of claim 9, further comprising classifying the subject with respect to likely benefit from a therapeutic regimen or lifestyle change.

15. The method of claim 9, further comprising accessing additional clinically relevant information about the subject.

16. The method of claim 9 wherein the at least one allele of the pattern 1 is selected from the group consisting of: the IL-1A (222/223) allele 4, IL-1A (gz5/gz6) allele 4, IL-1A (−889) allele 1, allele 3 of the gaat.p33330 marker of the IL-1 B/IL-1RN intergenic region, allele 3 of the Y31 marker of the IL-1B/IL-1RN intergenic region, IL-1RN (+2018) allele 2, IL-1 A (+4845) allele 1, IL-1B (+3954) allele 1, IL-1B (−3737) allele 1, and IL-1RN (VNTR) allele 2.

17. The method of claim 9 wherein the at least one allele of the pattern 2 is selected from the group consisting of: the IL-1A (222/223) allele 3, IL-1A (gz5/gz6) allele 3, IL-1A (−889) allele 2, allele 4 of the gaat.p33330 marker of the IL-1B/IL-1RN intergenic region, allele 6 of the Y31 marker of the IL-1B/IL-1RN intergenic region, IL-1RN (+2018) allele 1, IL-1A (+4845) allele 2, IL-1B (+3954) allele 2, IL-1B (−3737) allele 1, and IL-1RN (VNTR) allele 1.

18. The method of claim 9 wherein the at least one allele of the pattern 3 is selected from the group consisting of: the IL-1A (−889) allele 1, IL-1A (+4845) allele 1, IL-1B (−3737) allele 2, IL-1B (+3954) allele 1, IL-1RN (+2018) allele 1, and IL-1RN (VNTR) allele 1.

19. A method for predicting the potential for cell division of a subject's endothelial cells, comprising accessing the subject's genotype with respect to at least one allele of an IL-1 pattern 1, pattern 2 and/or pattern 3, wherein the presence or absence of the at least one allele of an IL-1 pattern 1, pattern 2 and/or pattern 3 provides information about the potential for cell division of the subject's endothelial cells.

20. The method of claim 19, wherein accessing a subject's genotype comprises obtaining previously known information about the subject's genotype.

21. The method of claim 19, wherein accessing the subject's genotype comprises:
(a) obtaining a nucleic acid sample from said subject; and
(b) detecting at least one allele of IL-1 pattern 1, pattern 2 and/or pattern 3.

22. The method of claim 19, wherein the method comprises accessing the subject's genotype comprises determining the genotype at the IL-1 RN (VNTR) locus.

23. A method for identifying a biomarker useful for determining an aging-related phenotype comprising,
(a) measuring a biomarker in at least one subject having an EOA-associated allele to obtain a first measured value; and
(b) measuring said biomarker in at least one subject having a non-EOA-associated genotype to obtain a second measured value,
wherein if said first measured value differs from said second measured value, said first measured value is an aging-related phenotype and said biomarker is a biomarker that is useful for determining an aging-related phenotype.

24. A method for screening test substances to identify a test substance that is likely to prevent or diminish the early onset or progression of an aging-related condition, the method comprising:
(a) observing a biomarker in a cell containing DNA comprised of at least one allele of pattern 1, pattern 2 and/or pattern 3 to obtain a first measured value of the biomarker;
(b) contacting the cell with a test substance; and
(c) observing the biomarker in the cell to obtain a second measured value of the biomarker, wherein a change in the measured value of the biomarker from a senescence-related phenotype to a non-senescence-related phenotype identifies a test substance that is likely to prevent or diminish the early onset or progression of aging-related diseases and conditions.

25. A method of claim 24 wherein the cells are human umbilical vein endothelial cells.

26. A method of claim 24 wherein the biomarker is selected from the following group: IL-1 B mRNA, IL-1A mRNA, IL-1RN mRNA, IL-1b protein, IL-1a protein, IL-1ra protein and cumulative cell population doublings in culture.

27. A method for screening genes to identify a gene that is likely to prevent or diminish the early onset or progression of an aging-related condition in a subject, said method comprising:
(a) contacting a cell containing DNA comprised of at least one allele of pattern 1, pattern 2 and/or pattern 3 with a test gene under conditions causing the test gene to enter one or more of the cells; and
(b) observing at least one biomarker in the subject, wherein a change in a biomarker from a senescence-related phenotype to a non-senescence-related phenotype identifies a test gene that is likely to prevent or diminish the early onset of aging-related diseases and conditions.

28. A method of claim 27 wherein said at least one biomarker is selected from the following group: IL-1B mRNA, IL-1A mRNA, IL-1RN mRNA, IL-1b protein, IL-1a protein, IL-1ra protein and cumulative cell population doublings in culture.

29. A cellular composition comprising cultured endothelial cells heterozygous for the IL-1 RN (VNTR) allele 2.

30. The cellular composition of claim 29, wherein the cultured endothelial cells are human unbilical endothelial cells.

31. A cellular composition comprising cultured endothelial cells homozygous for the IL-1 RN (VNTR) allele 2.

32. A method of preventing or diminishing the early onset or progression of an aging-related condition, the method comprising contacting a subject with a substance identified according to the method of claim 24, wherein contacting the subject with the substance prevents or diminishes the early onset of an aging-related condition.

33. A method of preventing or diminishing the early onset of an aging-related condition, the method comprising contacting a subject with a gene identified according to the method of claim 27, wherein contacting a subject with the gene prevents or diminishes the early onset of an aging-related condition.

34. A method of preventing or diminishing senescence of cultured cells, the method comprising contacting cells with a substance identified according to the method of claim 24, wherein contacting cells with the substance prevents or diminishes the senescence of the cultured cells.

35. A method of preventing or diminishing senescence of cultured cells, the method comprising contacting cells with a gene identified according to the method of claim 27, wherein contacting cells with said gene prevents or diminishes the senescence of the cultured cells.

36. A method of determining a subject's stage in an aging-related condition comprising,
(a) observing at least one biomarker identified according to the method of claim 22 in the subject; and
(b) determining the degree to which said biomarker evinces an aging-related phenotype, wherein the greater the degree to which the biomarker evinces an aging related-phenotype, the later the stage of the aging related condition.
